(19) United States
(12) Patent Application Publication
(10) Pub. No.: US 2025/0177422 A1
(43) Pub. Date: Jun. 5, 2025

(54) CO-ADMINISTRATION OF A CO-CRYSTAL OF PSILOCYBIN AND PSILOCIN WITH AN ATYPICAL ANTIPSYCHOTIC

(71) Applicant: ZYLORION HEALTH INC., Calgary (CA)

(72) Inventors: Peter Silverstone, Calgary (CA); Robert Laprairie, Saskatoon (CA); Deborah Kurrasch, Calgary (CA)

(21) Appl. No.: 18/942,043

(22) Filed: Nov. 8, 2024

Related U.S. Application Data
(60) Provisional application No. 63/597,415, filed on Nov. 9, 2023.

(51) Int. Cl. A61K 31/675; A61K 31/4045; A61K 31/496; A61K 33/00; A61K 45/06; A61P 25/28

(52) U.S. Cl. CPC A61K 31/675; A61K 31/4045; A61K 31/496; A61K 33/00; A61K 45/06; A61P 25/28

(57) ABSTRACT

The present disclosure provides a method of treating or ameliorating a mental health disorder or a central nervous system disorder in a subject in need thereof, comprising administering to the subject a composition comprising a crystalline form of psilocin and psilocybin, wherein the crystalline form is co-crystal form A of psilocin and psilocybin and an atypical antipsychotic.

### CROSS REFERENCE TO RELATED APPLICATIONS

This application claims benefit of priority under 35 U.S.C. § 119(e) of U.S. Provisional Application No. 63/597,415, filed on Nov. 9, 2023. The disclosure of the prior application is herein incorporated by reference in the disclosure of this application in its entirety.

### FIELD OF THE INVENTION

This invention is directed to the use of a co-crystal of psilocybin and psilocin along with an atypical antipsychotic in treating a mental health disorder or a central nervous system disorder in a subject in need thereof. The psilocybin and psilocin co-crystal may further be used to induce neuroplasticity and increase brain-derived neurotrophic factor (BDNF) levels, all with a lowered risk of cardiac adverse events.

### BACKGROUND INFORMATION

Various natural plants and fungi contain psychedelic compounds which cause changes in brain function or activity (‘neuroactive’ compounds) and at higher doses can also cause individuals to experience hallucinogenic or similar experiences (‘psychedelic’ compounds).

Psychedelic experiences have long been suggested to have potential therapeutic benefits, and more recent research has suggested that psychedelics may have benefits in a wide range of clinical conditions where other treatments have not worked. Examples of these include treatment-resistant major depressive disorder (TRD), post-traumatic stress disorder (PTSD), and addiction and abuse disorders. Such treatments usually involve a single administration of psychedelics followed by typical psychedelic experiences. For example, several resent recent studies with psilocybin have shown the beneficial clinical effects following a single dose of 25 mg sufficient to cause a robust psychedelic experience, accompanied by psychotherapeutic support for TRD.

More recently, chronic administration of very low doses (so-called ‘microdosing’) with psychedelics such as psilocybin and lysergic acid diethylamide (LSD) have also been suggested as helping individuals with a wide range of neuropsychiatric and central nervous system (CNS) disorders. These administrations are not accompanied by psychedelic experiences, but have been widely reported to have a variety of effects of overall psychological wellbeing and mental health. However, there has been far less research on the potential clinical benefits of longer-term microdosing, although there are a large number of anecdotal reports and poorly controlled studies purporting to demonstrate a wide range of benefits.

Examples of psychedelics contained in plants are N,N-dimethyltryptamine (DMT) found in Psychotria viridis and mescaline from the peyote cactus. Similarly, there are over 180 different species of fungi which contain the psychedelic psilocybin and its active metabolite psilocin. There are also a number of synthetic psychedelics, such as 3,4-Methylenedioxymethamphetamine (MDMA), 3,4-Methylenedioxyamphetamine (MDA), 2,5-Dimethoxy-4-ethoxyamphetamine (MEM), and lysergic acid diethylamide (LSD). The psychedelic effects of both psilocybin (via its active metabolite, psilocin) and LSD are often considered to represent ‘classic’ psychedelic experiences and include a wide range of perceptual experiences. This can include objects appearing more vivid, changes in auditory perception, changes in time perception, intense religious experiences, a sense of ‘oneness’, and mood changes such as a sense of euphoria or joy. Sometimes negative emotions are experienced such as severe anxiety, terror, or paranoia. Among the most common type of naturally occurring psychedelics is psilocybin, which has been found in over 180 species of mushroom globally. Psilocybin-containing mushrooms also have other neuroactive and psychedelic compounds, the most abundant of which is its primary metabolite, psilocin. In humans, psilocybin is metabolized in the gastrointestinal system, primarily to psilocin, which is then absorbed and causes the psychedelic effects.

In terms of a potential mechanism of action for clinical benefits, many psychedelic compounds have a structural similarity to serotonin (5-hydroxytrytamine, 5HT), and all of the aforementioned psychedelics, including psilocybin and psilocin, have been suggested to have their primary psychedelic actions via effects on 5HT2A receptors on the cell surface of CNS neurons. Recent research evidence has also shown that there are some intracellular 5HT2A receptors which may also play a role, although this is currently uncertain, but again the primary mechanism purported to explain the clinical benefits of psychedelics has been via their effects on 5HT2A receptors.

However, these well-recognized effects of psychedelics such as psilocin and psilocybin on 5HT2A receptors do not, by themselves, appear to fully explain the research and clinical findings seen with use of these substances. Thus, other drugs that affect 5HT2A receptors do not have the same clinical profile or range of clinical benefits as psychedelics.

Supporting this, recent research findings have suggested that the clinical effects of at least psilocin and LSD, in-part, are due to an increase in neuroplasticity which may occur via one or more mechanisms, and not simply by actions on the 5HT2A receptor.

More specifically, a neuroplasticity mechanism has been identified which involves allosteric modulation of the tyrosine receptor kinase B (TrkB) and increases the binding to this receptor of brain-derived neurotrophic factor (BDNF). This also increases the overall effectiveness of BDNF on brain plasticity including neurogenesis.

While psychedelics are putative treatments for mental health disorders, other drugs have been demonstrated effectiveness for over 70 years. One such group of drugs are antipsychotics, which are primarily used for the treatment of Schizophrenia and other psychotic disorders, and increasingly for the treatment of Bipolar Disorder and as an adjunctive treatment for Major Depressive Disorder (MDD). Their clinical benefits have been proposed to be primarily via actions which blockade the dopamine (D2) receptor (i.e. D2 receptor antagonism). However, problematically this same mechanism of action can cause major side-effects including inducing symptoms similar to Parkinson's Disease and other movement disorders.

For these reasons and to address these side-effects, newer antipsychotic drug compounds were developed, the first of which was clozapine in 1972. Because they do not have the same side-effects, they have been termed ‘atypical antipsychotics’. Their clinical benefits, with a reduction in movement disorders, is now believed to be due to impacts at central 5HT2A receptors which is believed to counter some of the side-effects induced by D2 receptor antagonism.

The atypical antipsychotic aripiprazole was approved for use by the Food and Drug Administration (FDA) in Schizophrenia in 2002 in doses of 10-30 mg/day, and then subsequently approved for the treatment of Bipolar Disorder in a similar dose range.

Aripiprazole was subsequently approved for use by the FDA as an adjunctive treatment for MDD in 2007, but in a lower approved dose range of 2-15 mg daily. This approval followed two Phase Three clinical trials which involved 743 patients whose depression had not responded adequately to an antidepressant. In an 8-week prospective trial either placebo or aripiprazole was then added to the antidepressant regimen for 6 weeks. The maximum dosage of aripiprazole when used in combination with fluoxetine or paroxetine was capped at 15 mg daily in consideration of the ability of these antidepressants to potentially increase aripiprazole levels via their inhibitory effect on the hepatic cytochrome isoenzyme CYP450-2D6.

The mechanism of action of aripiprazole nonetheless remains uncertain since its actions at both D2 and 5HT2A receptors are not sufficient to fully explain all of the clinical findings from this drug.

Thus, despite commonalities of action from both the psychedelics psilocin and psilocybin and the atypical antipsychotic aripiprazole, in that they have both been proposed to act on 5HT2A receptors, this is unlikely to fully explain their clinical benefits.

Additionally, early studies have suggested that both of these groups of compounds may have impacts of different aspects of neuroplasticity, although this is not currently widely accepted as the explanation for clinical benefits. There have been some research studies suggesting that atypical antipsychotics may have a few effects on some aspects of neuroplasticity, although this does not appear to be via the TrkB binding, BDNF levels or activity, or changes which measure neurogenesis specifically.

### SUMMARY

The present disclosure provides for a method of treating a mental health disorder or a central nervous system disorder, inducing neuroplasticity, stimulating neuronal growth, increasing the number of neuronal progenitor cells, increasing BDNF levels, or decreasing neuroinflammation in a subject in need thereof, comprising administering to the subject a crystalline form of psilocin and psilocybin:

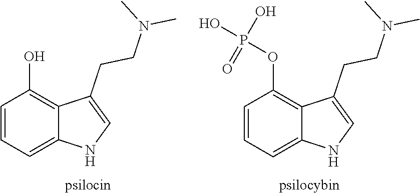

and an atypical antipsychotic.

In one embodiment, the crystalline form of psilocin and psilocybin is co-crystal Form A. Co-crystal Form A may be characterized by a XRPD pattern including a significant peak at a 2θ angle of about 10.1°. A XRPD pattern of co-crystal Form A may additionally include a significant peak at 2θ angle of about 19.16°. A XRPD pattern of co-crystal Form A may additionally include significant peaks at 2θ angle of about 10.74°, about 25.3°, and about 24.07°. Yet further, a XRPD pattern of co-crystal Form A may additionally include significant peaks at 2θ angle of about 14.54°, about 16.5°, about 13.44°, about 23.42°, or about 8.62°. In one embodiment, the crystalline form is co-crystal Form A. Co-crystal Form A may be characterized by a XRPD pattern including a significant peak at a 2θ angle of about 10.10 and about 19.16°. Co-crystal Form A may be characterized by a XRPD pattern including a significant peak at a 2θ angle of about 10.1°, about 19.16°, about 14.54°, about 16.5°, about 13.44°, about 23.42°, and about 8.62°.

The present disclosure provides for a method of treating a mental health disorder or a central nervous system disorder, inducing neuroplasticity, stimulating neuronal growth, increasing the number of neuronal progenitor cells, increasing BDNF levels, or decreasing neuroinflammation in a subject in need thereof, comprising administering to the subject a crystalline form of psilocin and psilocybin:

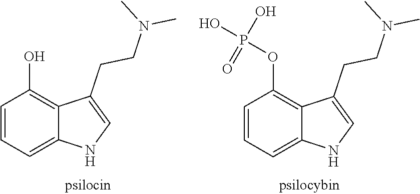

and an atypical antipsychotic;
wherein the crystalline form is co-crystal Fom A;
wherein co-crystal Form A is characterized by a XRPD pattern comprising a significant peak at a 2θ angle of about 10.1°, about 19.16°, about 14.54°, about 16.5°, about 13.44°, about 23.42°, and about 8.62°.

The present disclosure provides for a method of treating a mental health disorder or a central nervous system disorder, inducing neuroplasticity, stimulating neuronal growth, increasing the number of neuronal progenitor cells, increasing BDNF levels, or decreasing neuroinflammation in a subject in need thereof, comprising administering to the subject a crystalline form of psilocin and psilocybin:

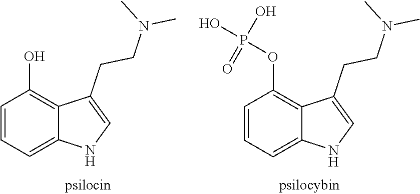

and an atypical antipsychotic;
wherein the crystalline form is co-crystal Fom A;
wherein co-crystal Form A is characterized by a XRPD pattern comprising a significant peak at a 2θ angle of about 10.10 and about 19.16°.

In one embodiment, the atypical antipsychotic is aripiprazole, clozapine, ziprasidone, cariprazine, iloperidone, quetiapine, lurasidone, paliperidone, amisulpride, olanzapine, risperidone, asenapine, clozaril, brexpiprazole, lumateperone, pimavanserin, or a combination thereof.

In one embodiment, the atypical antipsychotic is aripiprazole.

In one embodiment, the mental health disorder is mood disorders, depressive disorders, mood instability disorders, mixed anxiety and depressive disorder, bipolar disorders, anxiety disorders, generalized anxiety disorders, social anxiety disorders, phobic disorders, panic attack disorders, schizophrenia, psychosis, schizoaffective disorders, eating disorders, anorexia nervosa, bulimia nervosa, substance abuse disorders, addiction disorders, alcohol abuse or dependence, opiate abuse or dependence, cocaine abuse or dependence, mixed drug abuse or dependence, behavioral addictions, gambling disorder, trauma-related disorders, post-traumatic stress disorders, grief-related disorders, loss-related disorders, end-of-life related disorders, cancer disorders, attention deficit disorder (ADD), attention deficit and hyperactivity disorder (ADHD), personality disorders, obsessive compulsive disorders, or a combination thereof.

### DESCRIPTION OF THE DRAWINGS

FIG. 1 shows Ca²⁺ release (as a percentage of LSD) at the 5-HT2A receptor after treatment with psilocin, co-crystal Form A, and co-crystal Form A with aripiprazole.

FIG. 2 shows Ca²⁺ release (as a percentage of LSD) at the 5-HT2B receptor after treatment with psilocin, co-crystal Form A, and co-crystal Form A with aripiprazole.

FIG. 3 shows βarrestin2 recruitment (as a percentage of LSD) at the 5-HT2A receptor after treatment with psilocin, co-crystal Form A, and co-crystal Form A with aripiprazole.

FIG. 4 shows the change in mice behavior is a Y-maze test in animals given oral co-crystal form A, aripiprazole, and co-crystal form A+aripiprazole for 15 days.

FIG. 5 shows the change in brain levels of BDNF in mice brain 1 hour after acute treatment.

FIG. 6 shows the change in increased neuronal progenitor/stem cell levels in mice brain following 14-days of oral co-crystal form A (3 mg/kg) alone and in combination with aripiprazole (0.3 mg/kg).

FIG. 7 shows the change in increased neuronal activation in mice 21-days of oral co-crystal form A (3 mg/kg) alone and in combination with aripiprazole (0.3 mg/kg).

FIG. 8 shows the levels of various cytokine biomarkers of neuroinflammation 60 minutes after oral administration of co-crystal form A+aripiprazole.

FIG. 9 shows the levels of TGF-β1 at Day 1 and Day 21 after administration of co-crystal Form A (0.3 mg/kg), aripiprazole (0.03 mg/kg), and co-crystal Form A (0.3 mg/kg)+aripiprazole (0.03 mg/kg) compared to control.

### DETAILED DESCRIPTION

The present invention is based on the seminal discovery that co-crystals of psilocybin and psilocin may be useful for treating a mental health disorder or a central nervous system disorder in a subject in need thereof.

Before the present compositions and methods are described, it is to be understood that this invention is not limited to the particular processes, formulations, compositions, or methodologies described, as these may vary. It is also to be understood that the terminology used in the description is for the purpose of describing the particular versions or embodiments only and is not intended to limit the scope of embodiments herein which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of embodiments herein, the preferred methods, devices, and materials are now described. All publications mentioned herein are incorporated by reference in their entirety. Nothing herein is to be construed as an admission that embodiments herein are not entitled to antedate such disclosure by virtue of prior invention.

It must also be noted that as used herein and in the appended claims, the singular forms “a,” “an,” and “the” include plural reference unless the context clearly dictates otherwise.

The transitional term “comprising,” which is synonymous with “including,” “containing,” or “characterized by,” is inclusive or open-ended and does not exclude additional, unrecited elements or method steps.

As used herein, the term “consists of” or “consisting of” means that the composition, formulation or the method includes only the elements, steps, or ingredients specifically recited in the particular claimed embodiment or claim.

As used herein, the term “consisting essentially of” or “consists essentially of” means that the composition, formulation or the method includes only the elements, steps or ingredients specifically recited in the particular claimed embodiment or claim and may optionally include additional elements, steps or ingredients that do not materially affect the basic and novel characteristics of the particular embodiment or claim. For example, the only active ingredient(s) in the formulation or method that treats the specified condition (e.g., nutrient depletion) is the specifically recited therapeutic(s) in the particular embodiment or claim.

As used herein, two embodiments are “mutually exclusive” when one is defined to be something which is different from the other.

When ranges of values are disclosed, and the notation “from n1 . . . to n2” or “between n1 . . . and n2” is used, where n1 and n2 are the numbers, then unless otherwise specified, this notation is intended to include the numbers themselves and the range between them. This range may be integral or continuous between and including the end values. By way of example, the range “from 1° C. to 3° C.,” which is intended to include 1° C., 3° C., and everything in between to any number of significant figures (e.g., 1.255° C., 2.1° C., 2.9999° C., etc.).

As used herein, the term “about” means plus or minus 10% of the numerical value of the number with which it is being used. Therefore, about 50% means in the range of 45% to 55%. For example, “about 100° C.” means a temperature in the range of 90° C. to 110° C. For example, “a 2θ angle of about 10°” means a 2θ angle in the range of 9° to 11°.

The term “substantially free” or as used herein, alone or in combination, and is used interchangeably with, the term “substantially pure”, refers to a compound which is free from all other compounds within the limits of detection as measured by any means including nuclear magnetic resonance (NMR), gas chromatography/mass spectroscopy (GC/MS), or liquid chromatography/mass spectroscopy (LC/MS). In embodiments, substantially free may be less than about 1.0%, less than about 0.5%, less than about 0.4%, less than about 0.3%, less than about 0.2%, less than about 0.1%, less than about 0.05%, or less than about 0.01%.

### Method of Treatment

In one embodiment, the present invention provides a method of treating a mental health disorder or a central nervous system disorder, inducing neuroplasticity, increasing BDNF levels, or decreasing neuroinflammation in a subject in need thereof, comprising administering to the subject a crystalline form of psilocin and psilocybin and an atypical antipsychotic.

The present disclosure provides for a method of treating a mental health disorder or a central nervous system disorder, inducing neuroplasticity, increasing BDNF levels, or decreasing neuroinflammation in a subject in need thereof, comprising administering to the subject a crystalline form of psilocin and psilocybin:

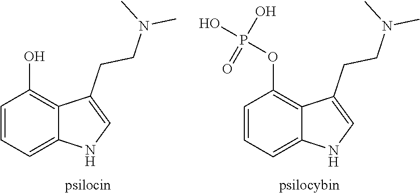

and an atypical antipsychotic.

In one embodiment, the crystalline form of psilocin and psilocybin is co-crystal Form A. Co-crystal Form A may be characterized by a XRPD pattern including a significant peak at a 2θ angle of about 10.1°. A XRPD pattern of co-crystal Form A may additionally include a significant peak at 2θ angle of about 19.16°. A XRPD pattern of co-crystal Form A may additionally include significant peaks at 2θ angle of about 10.74°, about 25.3°, and about 24.07°. Yet further, a XRPD pattern of co-crystal Form A may additionally include significant peaks at 2θ angle of about 14.54°, about 16.5°, about 13.44°, about 23.42°, or about 8.62°. In one embodiment, the crystalline form is co-crystal Form A. Co-crystal Form A may be characterized by a XRPD pattern including a significant peak at a 2θ angle of about 10.10 and about 19.16°. Co-crystal Form A may be characterized by a XRPD pattern including a significant peak at a 2θ angle of about 10.1°, about 19.16°, about 14.54°, about 16.5°, about 13.44°, about 23.42°, and about 8.62°.

In one embodiment, the present invention provides a method of treating a mental health disorder or a central nervous system disorder in a subject in need thereof, comprising administering to the subject a crystalline form of psilocin and psilocybin and an atypical antipsychotic, wherein the crystalline form is co-crystal Form A. In one embodiment, the co-crystal Form A Form A is characterized by a XRPD pattern comprising a significant peak at a 2θ angle of about 10.1°, about 19.16°, about 14.54°, about 16.5°, about 13.44°, about 23.42°, and about 8.62°. In one embodiment, the co-crystal Form A Form A is characterized by a XRPD pattern comprising a significant peak at a 2θ angle of about 10.1° and about 19.16°.

In one embodiment, the present invention provides a method inducing neuroplasticity in a subject in need thereof, comprising administering to the subject a crystalline form of psilocin and psilocybin and an atypical antipsychotic, wherein the crystalline form is co-crystal Form A. In one embodiment, the co-crystal Form A Form A is characterized by a XRPD pattern comprising a significant peak at a 2θ angle of about 10.1°, about 19.16°, about 14.54°, about 16.5°, about 13.44°, about 23.42°, and about 8.62°. In one embodiment, the co-crystal Form A Form A is characterized by a XRPD pattern comprising a significant peak at a 2θ angle of about 10.10 and about 19.16°.

In one embodiment, the present invention provides a method increasing BDNF levels in a subject in need thereof, comprising administering to the subject a crystalline form of psilocin and psilocybin and an atypical antipsychotic, wherein the crystalline form is co-crystal Form A. In one embodiment, the co-crystal Form A Form A is characterized by a XRPD pattern comprising a significant peak at a 2θ angle of about 10.10, about 19.16°, about 14.54°, about 16.5°, about 13.44°, about 23.42°, and about 8.62°. In one embodiment, the co-crystal Form A Form A is characterized by a XRPD pattern comprising a significant peak at a 2θ angle of about 10.1° and about 19.16°.

In one embodiment, the present invention provides a method of increasing decreasing neuroinflammation in a subject in need thereof, comprising administering to the subject a crystalline form of psilocin and psilocybin and an atypical antipsychotic, wherein the crystalline form is co-crystal Form A. In one embodiment, the co-crystal Form A Form A is characterized by a XRPD pattern comprising a significant peak at a 2θ angle of about 10.1°, about 19.16°, about 14.54°, about 16.5°, about 13.44°, about 23.42°, and about 8.62°. In one embodiment, the co-crystal Form A Form A is characterized by a XRPD pattern comprising a significant peak at a 2θ angle of about 10.1° and about 19.16°.

The terms “treat,” “treated,” “treating”, or “treatment” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total, whether induction of or maintenance of), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease and prolonging disease-free survival as compared to disease-free survival if not receiving treatment and prolonging disease-free survival as compared to disease-free survival if not receiving treatment.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., amelioration of symptoms of a mental health disorder or a central nervous system disorder). The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraarterial, intraorbital, intracardiac, intradermal, transdermal, subarachnoid, intraspinal, oral, sublingual, buccal, rectal, nasal administrations, as well infusion. Suitable unit dosage forms, include, but are not limited to powders, tablets, pills, capsules, lozenges, suppositories, patches, nasal sprays, injectables, implantable sustained-release formulations, lipid complexes, etc.

As used herein, the terms “acute administration” and “acute exposure” refer to refer to a single dose.

As used herein, the terms “chronic administration” and “chronic exposure” refer to a repeated daily administration for a minimum of 5 days. In some embodiments the co-crystal Form A is administered for 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, or 40 days. In some embodiments the co-crystal Form A is administered for 5 days. In some embodiments the co-crystal Form A is administered for 6 days. In some embodiments the co-crystal Form A is administered for 7 days. In some embodiments the co-crystal Form A is administered for 8 days, In some embodiments the co-crystal Form A is administered for 9 days. In some embodiments the co-crystal Form A is administered for 10 days. In some embodiments the co-crystal Form A is administered for 11 days. In some embodiments the co-crystal Form A is administered for 12 days. In some embodiments the co-crystal Form A is administered for 13 days. In some embodiments the co-crystal Form A is administered for 14 days. In some embodiments the co-crystal Form A is administered for 15 days. In some embodiments the co-crystal Form A is administered for 16 days. In some embodiments the co-crystal Form A is administered for 17 days. In some embodiments the co-crystal Form A is administered for 18 days. In some embodiments the co-crystal Form A is administered for 19 days. In some embodiments the co-crystal Form A is administered for 20 days. In some embodiments the co-crystal Form A is administered for 21 days, In some embodiments the co-crystal Form A is administered for 22 days, In some embodiments the co-crystal Form A is administered for 23 days. In some embodiments the co-crystal Form A is administered for 24 days. In some embodiments the co-crystal Form A is administered for 25 days. In some embodiments the co-crystal Form A is administered for 26 days. In some embodiments the co-crystal Form A is administered for 27 days. In some embodiments the co-crystal Form A is administered for 28 days. In some embodiments the co-crystal Form A is administered for 29 days. In some embodiments the co-crystal Form A is administered for 30 days. In some embodiments the co-crystal Form A is administered for 31 days. In some embodiments the co-crystal Form A is administered for 32 days. In some embodiments the co-crystal Form A is administered for 33 days. In some embodiments the co-crystal Form A is administered for 34 days. In some embodiments the co-crystal Form A is administered for 35 days. In some embodiments the co-crystal Form A is administered for 36 days. In some embodiments the co-crystal Form A is administered for 37 days. In some embodiments the co-crystal Form A is administered for 38 days. In some embodiments the co-crystal Form A is administered for 39 days. In some embodiments the co-crystal Form A is administered for 40 days.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

In some embodiments, the atypical antipsychotic is aripiprazole, clozapine, ziprasidone, cariprazine, iloperidone, quetiapine, lurasidone, paliperidone, amisulpride, olanzapine, risperidone, asenapine, clozaril, brexpiprazole, lumateperone, pimavanserin, or a combination thereof.

In some embodiments, the atypical antipsychotic is aripiprazole.

Mental health disorders, or mental illness, refer to a wide range of disorders that include, but are not limited to, mood disorders, depressive disorders, mood instability disorders, mixed anxiety and depressive disorder, bipolar disorders, anxiety disorders, generalized anxiety disorders, social anxiety disorders, phobic disorders, panic attack disorders, schizophrenia, psychosis, schizoaffective disorders, eating disorders, anorexia nervosa, bulimia nervosa, substance abuse disorders, addiction disorders, alcohol abuse or dependence, opiate abuse or dependence, cocaine abuse or dependence, mixed drug abuse or dependence, behavioral addictions, gambling disorder, trauma-related disorders, post-traumatic stress disorders, grief-related disorders, loss-related disorders, end-of-life related disorders, cancer disorders, attention deficit disorder (ADD), attention deficit and hyperactivity disorder (ADHD), personality disorders, and obsessive compulsive disorders, and any combinations of these. The full list of currently accepted mental health disorders is given in the Diagnostic and Statical Manual, 5th edition (DSM5) which is incorporated by reference herein in its entirety. The central nervous system (CNS) disorders include, but are not limited to, both psychological and physical trauma following burns, injury, or any other physical cause, post-amputation, post brain-injury, post-stroke, post heart attack, post-diabetes, chronic pain disorders, migraines, chronic headaches, chronic back pain, chronic arthritic pain, chronic pain from any cause, cognitive disorders, cognitive deterioration, Alzheimer's Disorder, dementia at any stage, dementia from any cause, age-associated memory impairment, neuropsychiatric disorders, fetal alcohol spectrum (FAS) disorders, autism spectrum disorders, multiple sclerosis, Parkinson's disease, Fragile X, Down's syndrome, fatigue, chronic fatigue, insomnia from any cause, and all post-covid syndromes. The severity of symptoms varies such that some individuals experience debilitating disease that precludes normal social function, while others suffer with intermittent repeated episodes across their lifespan. Although the presentation and diagnostic criteria among mental illness and CNS conditions are distinct in part, there are common endophenotypes of note across the diseases, and often comorbidities exist.

In some embodiments, the mental health disorder is mood disorders, depressive disorders, mood instability disorders, mixed anxiety and depressive disorder, bipolar disorders, anxiety disorders, generalized anxiety disorders, social anxiety disorders, phobic disorders, panic attack disorders, schizophrenia, psychosis, schizoaffective disorders, eating disorders, anorexia nervosa, bulimia nervosa, substance abuse disorders, addiction disorders, alcohol abuse or dependence, opiate abuse or dependence, cocaine abuse or dependence, mixed drug abuse or dependence, behavioral addictions, gambling disorder, trauma-related disorders, post-traumatic stress disorders, grief-related disorders, loss-related disorders, end-of-life related disorders, cancer disorders, attention deficit disorder (ADD), attention deficit and hyperactivity disorder (ADHD), personality disorders, obsessive compulsive disorders, or a combination thereof.

In some embodiments, the mental health disorder is mood disorders, mixed anxiety and depressive disorder, anxiety disorders, generalized anxiety disorders, social anxiety disorders, substance abuse disorders, addiction disorders, alcohol abuse or dependence, opiate abuse or dependence, cocaine abuse or dependence, mixed drug abuse or dependence, behavioral addictions, gambling disorder, post-traumatic stress disorders, or a combination thereof.

In some embodiments, the mental health disorder is mood disorders.

In some embodiments, the mental health disorder is mixed anxiety and depressive disorder.

In some embodiments, the mental health disorder is anxiety disorders.

In some embodiments, the mental health disorder is generalized anxiety disorders.

In some embodiments, the mental health disorder is social anxiety disorders.

In some embodiments, the mental health disorder is substance abuse disorders.

In some embodiments, the mental health disorder is addiction disorders.

In some embodiments, the mental health disorder is alcohol abuse or dependence.

In some embodiments, the mental health disorder is opiate abuse or dependence.

In some embodiments, the mental health disorder is cocaine abuse or dependence.

In some embodiments, the mental health disorder is mixed drug abuse or dependence.

In some embodiments, the mental health disorder is behavioral addictions.

In some embodiments, the mental health disorder is gambling disorder.

In some embodiments, the mental health disorder is post-traumatic stress disorders.

Depressive disorders are characterized by low mood, feeling sad or hopeless, increased irritability, sleep disturbance, lowered energy and feeling tired, poor concentration, lowered self-esteem or feeling worthless, lack of interest or pleasure in things, slowed movement, thinking they would be better off dead and self-harm, and actively considering and attempting suicide, and include major depressive disorder, bipolar depression, treatment resistant depression, and dysthymic disorder.

Anxiety disorders are characterized by feeling nervous, anxious, having difficulty breathing, avoiding people or activities, worrying excessively, physical symptoms including chest pain, palpitations of the heart, sweating, nausea, stomach ache, headache, neckache, poor sleep, poor concentration, sweating, and dizziness and include generalized anxiety disorder, social anxiety disorder, panic disorder, agoraphobia, phobias, and separation anxiety disorder.

Substance abuse and addiction disorders are identified when and individual has recurrent use of alcohol or drugs which cause significant impairment in issues negatively impacting health, work, school, and home, with the severity depending upon the type and frequency of substance use, social and interpersonal problems related to this use, impact on family, friends, work, school, and relationships, withdrawal symptoms, tolerance, unsuccessful attempts to stop, physical and mental health problems from this use, and cravings, and include alcohol use disorder, cannabis use disorder, hallucinogen use disorder, inhalant use disorder, opioid use disorder, sedative hypnotic or anxiolytic use disorder, stimulant use disorder, tobacco use disorder, unknown substance use disorder, with these disorders occurring either alone or in combination

Post-traumatic stress disorder (PTSD) is characterized by flashbacks, nightmares, involuntary memories, emotional distress or physical reactions after being reminded of the trauma, avoidance of anything that can trigger such remembrances, avoiding activities, social isolation, increased anger or aggression, increased irritability, participating in risky behavior, poor concentration, problems sleeping, and an increased startle response, which can further be described as those with dissociated experiences.

In some embodiments, the central nervous system disorder is Agraphia, Agnosia, Alzheimer's disease, Amnesia, Amyotrophic lateral sclerosis, Aneurysm, Anosognosia, Aphasia, Apraxia, Asomatognosia, Asperger syndrome, Ataxia, Attention deficit hyperactivity disorder, Auditory processing disorder, Autism spectrum disorder, Back pain, Bell's palsy, Bipolar disorder, Brain damage, Brachial plexus injury, Brain injury, Brain tumor, Canavan disease, Capgras delusion, Carpal tunnel syndrome, Causalgia, Central pain syndrome, Central pontine myelinolysis, Centronuclear myopathy, Cephalic disorder, Cerebral arteriosclerosis, Cerebral atrophy, Cerebral vasculitis, Cervical spinal stenosis, Chorea, Chronic fatigue syndrome, Chronic inflammatory demyelinating polyneuropathy, Chronic pain, Cluster headache, Complex post-traumatic stress disorder, Complex regional pain syndrome, Compression neuropathy, Cranial arteritis, Creutzfeldt-Jakob disease, Cumulative trauma disorders, Cushing's syndrome, Cyclic vomiting syndrome, Cyclothymic disorder, Dementia, Dermatillomania, Diabetic neuropathy, Diffuse sclerosis, Dysarthria, Dyscalculia, Dysphagia, Dysgraphia, Dyskinesia, Dyslexia, Dystonia, Encephalopathy, Epilepsy, Erythromelalgia, Essential tremor, Fetal alcohol syndrome, Febrile seizures, Fibromyalgia, Friedreich's ataxia, Frontotemporal dementia, Functional neurological symptom disorder, Gaucher's disease, Generalized anxiety disorder, Gray matter heterotopia, Guillain-Barr6 syndrome, Head injury, Headache, Hemifacial spasm, Huntington's disease, Hypoalgesia, Hypoesthesia, Inflammatory myopathy, Intracranial hypertension, Korsakoff Syndrome, Lewy body dementia, Lumbar disc disease, Lupus erythematosus—neurological sequelae, Lyme disease, Menieres disease, Migraine, Mild brain injury, Multi-infarct dementia, Multiple sclerosis, Muscular dystrophy, Myasthenia gravis, Myoclonus, Myopathy, Neuralgia, Neurofibromatosis, Neuromyotonia, Neuropathy, Niemann-Pick disease, Optic neuritis, Otosclerosis, Paraplegia, Paralysis, Paresthesia, Paresis, Parkinson's disease, Paraneoplastic diseases, Periodic paralyses, Peripheral neuropathy, Phantom limb pain, Pick's disease, Polyneuropathy, Postherpetic neuralgia, Posttraumatic stress disorder, Primary lateral sclerosis, Prion diseases, Progressive hemifacial atrophy, Progressive supranuclear palsy, Prosopagnosia, Radiculopathy, Reflex neurovascular dystrophy, Repetitive stress injury, Restless legs syndrome, Rett syndrome, Reye's syndrome, Sclerosis, Seizures, Sensory processing disorder, Sjagren's syndrome, Sleep apnoea, Spasticity, Spinal cord injury, Spinal cord tumors, Spinal muscular atrophy, Spinocerebellar ataxia, Stiff-person syndrome, Stroke, Sydenham's chorea, Traumatic encephalopathy, Tardive dyskinesia, Tarsal tunnel syndrome, Temporal arteritis, Temporal lobe epilepsy, Tinnitus, Tourette syndrome, Toxic encephalopathy, Transient ischemic attack, Transverse myelitis, Traumatic brain injury, Tremor, Trichotillomania, Trigeminal neuralgia, Vestibular schwannoma, Vertigo, Wernicke's encephalopathy, Wilson's disease, or a combination thereof.

While drugs that work as ‘atypical antipsychotics’ bind to dopaminergic D2 receptors, many of the atypical antipsychotics bind to other receptors at significantly greater potency (as measured by lower nM binding). Two of the common receptors where they are most potent include serotonin 5-HT2A and 5-HT2B receptors. There are multiple potential benefits of these drugs that have nothing to do with an ‘antipsychotic’ potential, particularly when they are given in low dose with combinations. More specifically, by being given in association with co-crystal form A, the atypical antipsychotic drug can block any potential negative impacts from impacts the co-crystal form A may itself have on serotonin 5-HT2A and 5-HT2B receptor. This may facilitate the effectiveness of co-crystal form A in a range of conditions, when given with low dose atypical antipsychotics, in a wide range of conditions for which the atypical antipsychotics do not themselves have specific therapeutic value. Examples include a wide range of neurological and immunological conditions.

In some embodiments, the central nervous system disorder is pain disorders, Alzheimer's disease, Parkinson's Disease, a cognitive disorder, or a combination thereof.

Chronic pain disorders are characterized by pain in one or more regions of the body including headache, low back pain, neck pain, arthritis pain, pain in context of cancer, neurogenic pain, psychogenic pain, and include conditions such as chronic arthritis, osteoarthritis, chronic fatigue syndrome, endometriosis, fibromyalgia, inflammatory bowel disease, interstitial cystitis, temporomandibular joint dysfunction, vulvodynia, cluster headaches, migraine, herpes zoster, frozen shoulder, complex regional pain syndrome, gout, post-surgical pain syndrome, prolapsed intervertebral disc, sciatica, and trigeminal neuralgia.

Cognitive disorders are characterized by poor functioning of the brain such that memory and other functions deteriorate including movement, speech, concentration and focus, problem-solving, analyzing sleep, tremors, tiredness, dizziness, headaches, hallucinations and confabulation, and include age-associated memory impairment, Alzheimer's disease, Picks disease, vascular dementia, post-stroke dementia, multi-infarct dementia, Parkinson's disease, frontal-lobe dementia, alcohol-induced dementia, and amnesia disorders including Korsakoff's syndrome.

In some embodiments, the method results in an improvement in one or more of neuroplasticity, calcium flux, 5-HT2A receptor activity, 5-HT1A, 5-HT7, TrkB activity, BDNF activity, neurogenesis, synaptogenesis, measures of second messenger functioning, or combinations thereof in the subject.

In some embodiments, the BDNF activity is increased.

In some embodiments, BDNF levels are increased by about 5% to about 150%, about 6% to about 150%, about 7% to about 150%, about 8% to about 150%, about 9% to about 150%, about 10% to about 150%, about 11% to about 150%, about 12% to about 150%, about 13% to about 150%, about 14% to about 150%, about 15% to about 150%, about 16% to about 150%, about 17% to about 150%, about 18% to about 150%, about 19% to about 150%, about 20% to about 150%, about 21% to about 150%, about 22% to about 150%, about 23% to about 150%, about 24% to about 150%, about 25% to about 150%, about 26% to about 150%, about 27% to about 150%, about 28% to about 150%, about 29% to about 150%, about 30% to about 150%, about 31% to about 150%, about 32% to about 150%, about 33% to about 150%, about 34% to about 150%, about 35% to about 150%, about 36% to about 150%, about 37% to about 150%, about 38% to about 150%, about 39% to about 150%, about 40% to about 150%, about 41% to about 150%, about 42% to about 150%, about 43% to about 150%, about 44% to about 150%, about 45% to about 150%, about 46% to about 150%, about 47% to about 150%, about 48% to about 150%, about 49% to about 150%, about 50% to about 150%, about 51% to about 150%, about 52% to about 150%, about 53% to about 150%, about 54% to about 150%, about 55% to about 150%, about 56% to about 150%, about 57% to about 150%, about 58% to about 150%, about 59% to about 150%, about 60% to about 150%, about 61% to about 150%, about 62% to about 150%, about 63% to about 150%, about 64% to about 150%, about 65% to about 150%, about 66% to about 150%, about 67% to about 150%, about 68% to about 150%, about 69% to about 150%, about 70% to about 150%, about 71% to about 150%, about 72% to about 150%, about 73% to about 150%, about 74% to about 150%, about 75% to about 150%, about 76% to about 150%, about 77% to about 150%, about 78% to about 150%, about 79% to about 150%, about 80% to about 150%, about 81% to about 150%, about 82% to about 150%, about 83% to about 150%, about 84% to about 150%, about 85% to about 150%, about 86% to about 150%, about 87% to about 150%, about 88% to about 150%, about 89% to about 150%, about 90% to about 150%, about 91% to about 150%, about 92% to about 150%, about 93% to about 150%, about 94% to about 150%, about 95% to about 150%, about 96% to about 150%, about 97% to about 150%, about 98% to about 150%, about 99% to about 150%, about 100% to about 150%, about 101% to about 150%, about 102% to about 150%, about 103% to about 150%, about 104% to about 150%, about 105% to about 150%, about 106% to about 150%, about 107% to about 150%, about 108% to about 150%, about 109% to about 150%, about 110% to about 150%, about 111% to about 150%, about 112% to about 150%, about 113% to about 150%, about 114% to about 150%, about 115% to about 150%, about 116% to about 150%, about 117% to about 150%, about 118% to about 150%, about 119% to about 150%, about 120% to about 150%, about 121% to about 150%, about 122% to about 150%, about 123% to about 150%, about 124% to about 150%, about 125% to about 150%, about 126% to about 150%, about 127% to about 150%, about 128% to about 150%, about 129% to about 150%, about 130% to about 150%, about 131% to about 150%, about 132% to about 150%, about 133% to about 150%, about 134% to about 150%, about 135% to about 150%, about 136% to about 150%, about 137% to about 150%, about 138% to about 150%, about 139% to about 150%, about 140% to about 150%, about 141% to about 150%, about 142% to about 150%, about 143% to about 150%, about 144% to about 150%, about 145% to about 150%, about 146% to about 150%, about 147% to about 150%, about 148% to about 150%, about 149% to about 150%, about 5% to about 149%, about 5% to about 148%, about 5% to about 147%, about 5% to about 146%, about 5% to about 145%, about 5% to about 144%, about 5% to about 143%, about 5% to about 142%, about 5% to about 141%, about 5% to about 140%, about 5% to about 139%, about 5% to about 138%, about 5% to about 137%, about 5% to about 136%, about 5% to about 135%, about 5% to about 134%, about 5% to about 133%, about 5% to about 132%, about 5% to about 131%, about 5% to about 130%, about 5% to about 129%, about 5% to about 128%, about 5% to about 127%, about 5% to about 126%, about 5% to about 125%, about 5% to about 124%, about 5% to about 123%, about 5% to about 122%, about 5% to about 121%, about 5% to about 120%, about 5% to about 119%, about 5% to about 118%, about 5% to about 117%, about 5% to about 116%, about 5% to about 115%, about 5% to about 114%, about 5% to about 113%, about 5% to about 112%, about 5% to about 111%, about 5% to about 110%, about 5% to about 109%, about 5% to about 108%, about 5% to about 107%, about 5% to about 106%, about 5% to about 105%, about 5% to about 104%, about 5% to about 103%, about 5% to about 102%, about 5% to about 101%, about 5% to about 100%, about 5% to about 99%, about 5% to about 98%, about 5% to about 97%, about 5% to about 96%, about 5% to about 95%, about 5% to about 94%, about 5% to about 93%, about 5% to about 92%, about 5% to about 91%, about 5% to about 90%, about 5% to about 89%, about 5% to about 88%, about 5% to about 87%, about 5% to about 86%, about 5% to about 85%, about 5% to about 84%, about 5% to about 83%, about 5% to about 82%, about 5% to about 81%, about 5% to about 80%, about 5% to about 79%, about 5% to about 78%, about 5% to about 77%, about 5% to about 76%, about 5% to about 75%, about 5% to about 74%, about 5% to about 73%, about 5% to about 72%, about 5% to about 71%, about 5% to about 70%, about 5% to about 69%, about 5% to about 68%, about 5% to about 67%, about 5% to about 66%, about 5% to about 65%, about 5% to about 64%, about 5% to about 63%, about 5% to about 62%, about 5% to about 61%, about 5% to about 60%, about 5% to about 59%, about 5% to about 58%, about 5% to about 57%, about 5% to about 56%, about 5% to about 55%, about 5% to about 54%, about 5% to about 53%, about 5% to about 52%, about 5% to about 51%, about 5% to about 50%, about 5% to about 49%, about 5% to about 48%, about 5% to about 47%, about 5% to about 46%, about 5% to about 45%, about 5% to about 44%, about 5% to about 43%, about 5% to about 42%, about 5% to about 41%, about 5% to about 40%, about 5% to about 39%, about 5% to about 38%, about 5% to about 37%, about 5% to about 36%, about 5% to about 35%, about 5% to about 34%, about 5% to about 33%, about 5% to about 32%, about 5% to about 31%, about 5% to about 30%, about 5% to about 29%, about 5% to about 28%, about 5% to about 27%, about 5% to about 26%, about 5% to about 25%, about 5% to about 24%, about 5% to about 23%, about 5% to about 22%, about 5% to about 21%, about 5% to about 20%, about 5% to about 19%, about 5% to about 18%, about 5% to about 17%, about 5% to about 16%, about 5% to about 15%, about 5% to about 14%, about 5% to about 13%, about 5% to about 12%, about 5% to about 11%, about 5% to about 10%, about 5% to about 9%, about 5% to about 8%, about 5% to about 7%, or about 5% to about 6%.

In some embodiments, the BDNF levels are increased by about 5% to about 75%.

In some embodiments, BDNF levels are increased by about 5% to about 150%.

In some embodiments, BDNF levels are increased by about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 122%, about 123%, about 124%, about 125%, about 126%, about 127%, about 128%, about 129%, about 130%, about 131%, about 132%, about 133%, about 134%, about 135%, about 136%, about 137%, about 138%, about 139%, about 140%, about 141%, about 142%, about 143%, about 144%, about 145%, about 146%, about 147%, about 148%, about 149%, about 150%, or a range between any two of these values.

In some embodiments, the method further comprises stimulating neurogenesis or neurite growth.

In some embodiments, stimulating neurogenesis or neurite growth comprises an increase in neuronal length.

In some embodiments, the increase in neuronal growth results in an increase of neurite length of about 100 μM to about 300 μM, about 110 μM to about 300 μM, about 120 μM to about 300 μM, about 130 μM to about 300 μM, about 140 μM to about 300 μM, about 150 μM to about 300 μM, about 160 μM to about 300 μM, about 170 μM to about 300 μM, about 180 μM to about 300 μM, about 190 μM to about 300 μM, about 200 μM to about 300 μM, about 210 μM to about 300 μM, about 220 μM to about 300 μM, about 230 μM to about 300 μM, about 240 μM to about 300 μM, about 250 μM to about 300 μM, about 260 μM to about 300 μM, about 270 μM to about 300 μM, about 280 μM to about 300 μM, about 290 μM to about 300 μM, about 100 μM to about 290 μM, about 100 μM to about 280 μM, about 100 μM to about 270 μM, about 100 μM to about 260 μM, about 100 μM to about 250 μM, about 100 μM to about 240 μM, about 100 μM to about 230 μM, about 100 μM to about 220 μM, about 100 μM to about 210 μM, about 100 μM to about 200 μM, about 100 μM to about 190 μM, about 100 μM to about 180 μM, about 100 μM to about 170 μM, about 100 μM to about 160 μM, about 100 μM to about 150 μM, about 100 μM to about 140 μM, about 100 μM to about 130 μM, or about 100 μM to about 110 μM.

In some embodiments, the increase in neuronal growth results in an increase of neurite length of about 100 μM to about 300 μM.

In some embodiments, the increase in neuronal growth results in an increase of neurite length of about 100 μM, about 110 μM, about 120 μM, about 130 μM, about 140 μM, about 150 μM, about 160 μM, about 170 μM, about 190 μM, about 190 μM, about 200 μM, about 210 μM, about 220 μM, about 230 μM, about 240 μM, about 250 μM, about 260 μM, about 270 μM, about 290 μM, about 290 μM, about 300 μM, or a range between any two of these values.

In some embodiments, the increase in neuronal growth results in an increase of neurite length of about 20% to about 100%, about 25% to about 100%, about 30% to about 100%, about 35% to about 100%, about 40% to about 100%, about 45% to about 100%, about 50% to about 100%, about 55% to about 100%, about 60% to about 100%, about 65% to about 100%, about 70% to about 100%, about 75% to about 100%, about 80% to about 100%, about 85% to about 100%, about 90% to about 100%, about 95% to about 100%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, or about 20% to about 25%.

In some embodiments, the increase in neuronal growth results in an increase of neurite length of about 20% to about 100%.

In some embodiments, the increase in neuronal growth results in an increase of neurite length of about 20%, about 25%, about 30%, about 35%, about 40%, about 45%, about 50%, about 55%, about 60%, about 65%, about 70%, about 75%, about 80%, about 85%, about 90%, about 95%, about 100%, or a range between any two of these values.

In some embodiments, the method comprises a decreased expression of a neuroinflammatory cytokine biomarker.

Inflammation is part of the biological response of body tissues to harmful stimuli. However, left unchecked, can damage healthy cells, tissues and organs, and may cause internal scarring, tissue death and damage to the DNA in previously healthy cells can be determined by measuring expression levels of a neuroinflammatory biomarker.

In some embodiments, the neuroinflammatory cytokine biomarker is Eotaxin, G-CSF, GM-CSF, IFNg, IL-1a, IL-1b, IL-2, IL-3, IL-4, IL-5, IL-6, IL-7, IL-9, IL-10, IL-12p40, IL-12p70, IL-13, IL-15, IL-17, IP-10, KC, LIF, LIX, M-CSF, MCP-1, MIG, MIP-1a, MIP-1b, MIP-2, RANTES, TNFa, VEGF, or a combination thereof.

In some embodiments, the neuroinflammatory cytokine biomarker is Eotaxin.

In some embodiments, the neuroinflammatory cytokine biomarker is G-CSF.

In some embodiments, the neuroinflammatory cytokine biomarker is GM-CSF.

In some embodiments, the neuroinflammatory cytokine biomarker is IFNγ.

In some embodiments, the neuroinflammatory cytokine biomarker is IL-1α.

In some embodiments, the neuroinflammatory cytokine biomarker is IL-1β.

In some embodiments, the neuroinflammatory cytokine biomarker is IL-2.

In some embodiments, the neuroinflammatory cytokine biomarker is IL-3.

In some embodiments, the neuroinflammatory cytokine biomarker is IL-4.

In some embodiments, the neuroinflammatory cytokine biomarker is IL-5.

In some embodiments, the neuroinflammatory cytokine biomarker is IL-6.

In some embodiments, the neuroinflammatory cytokine biomarker is IL-7.

In some embodiments, the neuroinflammatory cytokine biomarker is IL-9.

In some embodiments, the neuroinflammatory cytokine biomarker is IL-10.

In some embodiments, the neuroinflammatory cytokine biomarker is IL-12p40.

In some embodiments, the neuroinflammatory cytokine biomarker is IL-12p70.

In some embodiments, the neuroinflammatory cytokine biomarker is IL-13.

In some embodiments, the neuroinflammatory cytokine biomarker is IL-15.

In some embodiments, the neuroinflammatory cytokine biomarker is 11-17.

In some embodiments, the neuroinflammatory cytokine biomarker is IP-10.

In some embodiments, the neuroinflammatory cytokine biomarker is KC.

In some embodiments, the neuroinflammatory cytokine biomarker is LIF.

In some embodiments, the neuroinflammatory cytokine biomarker is LIX.

In some embodiments, the neuroinflammatory cytokine biomarker is M-CSF.

In some embodiments, the neuroinflammatory cytokine biomarker is MCP-1.

In some embodiments, the neuroinflammatory cytokine biomarker is MIG.

In some embodiments, the neuroinflammatory cytokine biomarker is MIP-1α.

In some embodiments, the neuroinflammatory cytokine biomarker is MIP-1β.

In some embodiments, the neuroinflammatory cytokine biomarker is MIP-2.

In some embodiments, the neuroinflammatory cytokine biomarker is RANTES.

In some embodiments, the neuroinflammatory cytokine biomarker is TNFα.

In some embodiments, the neuroinflammatory cytokine biomarker is VEGF.

In some embodiments, the neuroinflammatory cytokine biomarker is decreased in expression by about 1% to about 80%, about 2% to about 80%, about 3% to about 80%, about 4% to about 80%, about 5% to about 80%, about 6% to about 80%, about 7% to about 80%, about 8% to about 80%, about 9% to about 80%, about 10% to about 80%, about 11% to about 80%, about 12% to about 80%, about 13% to about 80%, about 14% to about 80%, about 15% to about 80%, about 16% to about 80%, about 17% to about 80%, about 18% to about 80%, about 19% to about 80%, about 20% to about 80%, about 21% to about 80%, about 22% to about 80%, about 23% to about 80%, about 24% to about 80%, about 25% to about 80%, about 26% to about 80%, about 27% to about 80%, about 28% to about 80%, about 29% to about 80%, about 30% to about 80%, about 31% to about 80%, about 32% to about 80%, about 33% to about 80%, about 34% to about 80%, about 35% to about 80%, about 36% to about 80%, about 37% to about 80%, about 38% to about 80%, about 39% to about 80%, about 40% to about 80%, about 41% to about 80%, about 42% to about 80%, about 43% to about 80%, about 44% to about 80%, about 45% to about 80%, about 46% to about 80%, about 47% to about 80%, about 48% to about 80%, about 49% to about 80%, about 50% to about 80%, about 51% to about 80%, about 52% to about 80%, about 53% to about 80%, about 54% to about 80%, about 55% to about 80%, about 56% to about 80%, about 57% to about 80%, about 58% to about 80%, about 59% to about 80%, about 60% to about 80%, about 61% to about 80%, about 62% to about 80%, about 63% to about 80%, about 64% to about 80%, about 65% to about 80%, about 66% to about 80%, about 67% to about 80%, about 68% to about 80%, about 69% to about 80%, about 70% to about 80%, about 71% to about 80%, about 72% to about 80%, about 73% to about 80%, about 74% to about 80%, about 75% to about 80%, about 76% to about 80%, about 77% to about 80%, about 78% to about 80%, about 79% to about 80%, about 1% to about 79%, about 1% to about 78%, about 1% to about 77%, about 1% to about 76%, about 1% to about 75%, about 1% to about 74%, about 1% to about 73%, about 1% to about 72%, about 1% to about 71%, about 1% to about 70%, about 1% to about 69%, about 1% to about 68%, about 1% to about 67%, about 1% to about 66%, about 1% to about 65%, about 1% to about 64%, about 1% to about 63%, about 1% to about 62%, about 1% to about 61%, about 1% to about 60%, about 1% to about 59%, about 1% to about 58%, about 1% to about 57%, about 1% to about 56%, about 1% to about 55%, about 1% to about 54%, about 1% to about 53%, about 1% to about 52%, about 1% to about 51%, about 1% to about 50%, about 1% to about 49%, about 1% to about 48%, about 1% to about 47%, about 1% to about 46%, about 1% to about 45%, about 1% to about 44%, about 1% to about 43%, about 1% to about 42%, about 1% to about 41%, about 1% to about 40%, about 1% to about 39%, about 1% to about 38%, about 1% to about 37%, about 1% to about 36%, about 1% to about 35%, about 1% to about 34%, about 1% to about 33%, about 1% to about 32%, about 1% to about 31%, about 1% to about 30%, about 1% to about 29%, about 1% to about 28%, about 1% to about 27%, about 1% to about 26%, about 1% to about 25%, about 1% to about 24%, about 1% to about 23%, about 1% to about 22%, about 1% to about 21%, about 1% to about 20%, about 1% to about 19%, about 1% to about 18%, about 1% to about 17%, about 1% to about 16%, about 1% to about 15%, about 1% to about 14%, about 1% to about 13%, about 1% to about 12%, about 1% to about 11%, about 1% to about 10%, about 1% to about 9%, about 1% to about 8%, about 1% to about 7%, about 1% to about 6%, about 1% to about 5%, about 1% to about 4%, about 1% to about 3%, or about 1% to about 2%.

In some embodiments, the neuroinflammatory cytokine biomarker is decreased in expression by about 1% to about 80%.

In some embodiments, the neuroinflammatory cytokine biomarker is decreased in expression by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, or a range between any two of these values.

In some embodiments, the neuroinflammatory cytokine biomarker is decreased after acute exposure to a crystalline form of psilocin and psilocybin.

In some embodiments, the neuroinflammatory cytokine biomarker is decreased after chronic exposure to a crystalline form of psilocin and psilocybin.

In some embodiments, the method decreases the risk of cardiovascular adverse events.

The 5HT2B receptor is present in cardiac tissue and longer-term stimulation can in some individuals potentially increase the risk of valvular heart disease (VHD), an effect which has been known for the past 20 years.

Because of this effect, clinical use of drugs which contain 5HT2B receptor agonist activity is often reduced or avoided in an attempt to mitigate this risk. This finding potentially limits the utility of psychedelics for clinical treatment. Methods to reduce this risk may thus significantly add to the potential clinical benefits of longer-term administration of psychedelics.

One conceivable mechanism to achieve a reduction in risk is by combining drugs which may have different effects on the 5HT2B, such as theoretical combination of both an agonist and an antagonist. The actual impact of this, however, is not possible to know in advance since it is impossible to predict the overall impact of drugs which have different binding profiles on the same receptor, so the overall impact following combining drugs with different effects on the 5HT2B receptors cannot be accurately predicted. This is particularly the case when drugs are not direct agonists, but act indirectly, where it is impossible to predict how any two drugs may act together given this level of uncertainty since both the agonist and inverse agonist are trying to bind to the same receptor at the same time.

Cardiovascular adverse events may result from administration of the Form A co-crystal. Cardiovascular adverse events include (ventricular heart disease, cardiac fibrosis, stroke, myocardial infarction, cardiovascular death, heart failure, ischemic cardiovascular events, etc).

In some embodiments, the decreased risk of cardiovascular adverse events comprises decreasing the risk of ventricular heart disease, cardiac fibrosis, or a combination thereof.

In some embodiments, the decreased risk of cardiovascular adverse events comprises maintaining plasma levels of TGF-β1.

Transforming growth factor beta (TGF-β) is a multifunctional cytokine belonging to the transforming growth factor superfamily that includes three different mammalian isoforms (TGF-β1, TGF-β2, and TGF-β3). TGF-β may impact levels of acetylcholine, slow-wave sleep, muscle regeneration, bone density, red blood cell formation, lymphocytes (T and B cells), cytotoxic T Cell (CD8), Natural Killer cell activity, macrophages activity, inflammatory response, tissue growth, wound healing, new blood vessel formation (angiogenesis), local inflammation and fibrosis, extracellular matrix deposition, and cognitive function.

TGF-β isoforms are upregulated and activated in myocardial diseases and have an important role in cardiac repair and remodeling, regulating the phenotype and function of cardiomyocytes, fibroblasts, immune cells and vascular cells. Cardiac injury triggers the generation of bioactive TGF-β from latent stores, through mechanisms involving proteases, integrins and specialized extracellular matrix (ECM) proteins. Changes in the plasma levels of TGF-β isoforms is often indicative of risk of cardiovascular injury and adverse effects.

In some embodiments, the atypical antipsychotic is administered simultaneously with the crystalline form.

In some embodiments, the atypical antipsychotic is administered sequentially with the crystalline form.

In some embodiments, the crystalline form is administered first.

In some embodiments, the atypical antipsychotic is administered first.

In some embodiments, the method further comprises administering an additional pharmaceutical agent.

In some embodiments, the additional pharmaceutical agent is a psychedelic compound, a mood stabilizer, an antidepressant, an anti-epileptic agent, an anti-anxiety agent, or a combination thereof.

In some embodiments, the additional pharmaceutical agent is a psychedelic compound.

In some embodiments, the psychedelic compound is selected from psilocybin, psilocin, LSD, MDMA, MDA, MEM, DMT, or a combination thereof.

In some embodiments, the additional pharmaceutical agent is a mood stabilizer.

In some embodiments, the mood stabilizer is lithium.

In some embodiments, the additional pharmaceutical agent is an antidepressant.

In some embodiments, the additional pharmaceutical agent is an anti-epileptic agent.

some embodiments, the additional pharmaceutical agent is an anti-anxiety agent.

In some embodiments, administering additional pharmaceutical agent decreases the risk of cardiovascular adverse events.

In some embodiments, the decreased risk of cardiovascular adverse events comprises decreasing the risk of ventricular heart disease, cardiac fibrosis, or a combination thereof.

In some embodiments, the additional pharmaceutical agent is administered simultaneously with co-crystal form A and the atypical antipsychotic.

In some embodiments, the additional pharmaceutical agent is administered sequentially with the co-crystal form A and the atypical antipsychotic.

In some embodiments, the co-crystal form A is administered first, the atypical antipsychotic is administered second, and the additional pharmaceutical agent is administered third.

In some embodiments, the co-crystal form A is administered first, the additional pharmaceutical agent is administered second, and the atypical antipsychotic is administered third.

In some embodiments, the atypical antipsychotic is administered first, co-crystal form A is administered second, and the additional pharmaceutical agent is administered third.

In some embodiments, the atypical antipsychotic is administered first, the additional pharmaceutical agent is administered second, and the co-crystal form A is administered third.

In some embodiments, the additional pharmaceutical agent is administered first, the crystalline form is administered second, and the atypical antipsychotic is administered third.

In some embodiments, the additional pharmaceutical agent is administered first, the atypical antipsychotic is administered second, and the crystalline form is administered third.

In one embodiment the crystalline form is co-crystal Form A, co-crystal Form B, or co-crystal Form C.

In one embodiment the crystalline form is co-crystal Form A.

In one embodiment, co-crystal Form A is characterized by a XRPD pattern comprising a significant peak at a 2θ angle of about 10.1°; the XRPD pattern further comprises a significant peak at a 2θ angle of about 19.16°; at a 2θ angle of about 10.74°, about 25.3°, and about 24.07°; and/or the XRPD pattern further comprises a significant peak at a 2θ angle of about 14.54°, about 16.5°, about 13.44°, about 23.42°, and about 8.62°. In one embodiment, the XRPD pattern further comprises a significant peak at a 2θ angle of about 10.74°, about 25.3°, about 24.07°, about 14.54°, about 16.5°, about 13.44°, about 23.42°, or about 8.62°. In one embodiment, co-crystal Form A is characterized by a DSC plot comprising a sharp endothermic event at a temperature of about 255° C. In one embodiment, co-crystal Form A has a ratio of psilocybin to psilocin of about 1:1. In one embodiment, co-crystal Form A has a chemical purity of about 95% or greater. In some embodiments, co-crystal Form A contains not more than about 5 mol % of other solid forms. In some embodiments, co-crystal Form A of psilocin and psilocybin comprises about 95 mol % co-crystal Form A, about 2.5 mol % psilocin, and about 2.5 mol % psilocybin. In one embodiment, co-crystal Form A is a co-crystal formed between psilocin and psilocybin. In one embodiment, co-crystal Form A is a salt formed between psilocin and psilocybin.

In one embodiment, co-crystal Form A is characterized by unit cell parameters which are substantially equal to the following:

### Unit Cell Dimensions:

a=9.3674(3) A
b=11.2660(6) A
c=24.2741(9) A
α=90 degrees
β=90 degrees
γ=90 degrees
Space group=P2₁2₁2₁
Molecules/asymmetric unit=1

In one embodiment, the unit cell parameters were measured about 296K. In one embodiment, co-crystal Form A has a geometry of hydrogen bonds substantially as listed in Table A. In one embodiment, co-crystal Form A has atomic coordinates of non-hydrogen atoms substantially as listed in Table B. In one embodiment, co-crystal Form A has atomic coordinates of hydrogen atoms substantially as listed in Table C. In one embodiment, co-crystal Form A is a co-crystal formed between psilocin and psilocybin. In one embodiment, co-crystal Form A is a salt formed between psilocin and psilocybin.

In one embodiment the crystalline form is co-crystal Form B.

In one embodiment, co-crystal Form B is characterized by a XRPD pattern comprising a significant peak at a 2θ angle of about 18.54°; the XRPD pattern further comprises a significant peak at a 2θ angle of about 8.54°; the XRPD pattern further comprises a significant peak at a 2θ angle of about 22.78°, about 14.27°, and about 21.120 and/or the XRPD pattern further comprises a significant peak at a 2θ angle of about 14.12°, about 10.05°, about 9.94°, about 24.94°, and about 25.02°. In one embodiment, co-crystal Form B is characterized by a DSC plot comprising a broad endothermic event at a temperature of about 168.2° C. In one embodiment, co-crystal Form B has ratio of psilocybin to psilocin of about 1.3:1. In one embodiment, co-crystal Form B has ratio of psilocybin to psilocin of about 1:1. In one embodiment, co-crystal Form B has a chemical purity of about 95% or greater. In some embodiments, co-crystal Form B contains not more than about 5 mol % of other solid forms. In some embodiments, co-crystal Form B of psilocin and psilocybin comprises about 92 mol % co-crystal Form B, about 2.5 mol % psilocin, and about 2.5 mol % psilocybin. In one embodiment, co-crystal Form B is a co-crystal formed between psilocin and psilocybin. In one embodiment, co-crystal Form B is a salt formed between psilocin and psilocybin.

In one embodiment the crystalline form is co-crystal Form C.

In one embodiment, co-crystal Form C is characterized by a XRPD pattern comprising a significant peak at a 2θ angle of about 9.18°; the XRPD pattern further comprises a significant peak at a 2θ angle of about 17.95°; the XRPD pattern further comprises a significant peak at a 2θ angle of about 10.42°, about 24.22°, and about 18.38°; and/or the XRPD pattern further comprises a significant peak at a 2θ angle of about 19.82°, about 17.46°, about 14.82°, about 22.38°, and about 14.06°. In one embodiment, co-crystal Form C is characterized by a DSC plot comprising a broad endothermic event at a temperature of about 25-140° C., with a peak at 98.7° C. In one embodiment, co-crystal Form C has ratio of psilocybin to psilocin of about 1:1. In one embodiment, co-crystal Form C has a chemical purity of about 95% or greater. In some embodiments, co-crystal Form C contains not more than about 5 mol % of other solid forms. In some embodiments, co-crystal Form C of psilocin and psilocybin comprises about 95 mol % co-crystal Form C, about 2.5 mol % psilocin, and about 2.5 mol % psilocybin. In one embodiment, co-crystal Form C is a co-crystal formed between psilocin and psilocybin. In one embodiment, co-crystal Form C is a salt formed between psilocin and psilocybin.

### Crystalline Forms

Crystalline form of psilocin and psilocybin are further elaborated below:

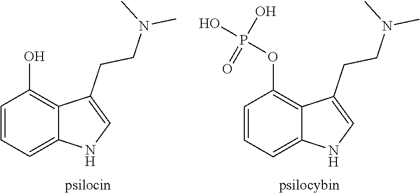

Co-crystal Form A, Form B, and Form C of psilocin and psilocybin are disclosed in WO2024/003610 A1, which is hereby incorporated by reference in its entirety.

In some embodiments, the crystalline form psilocin and psilocybin is co-crystal Form A.

In some embodiments, co-crystal Form A is characterized by a XRPD pattern comprising a significant peak at a 2θ angle of about 10.1°. In some embodiments, the XRPD pattern of co-crystal Form A may additionally comprise a significant peak at 2θ angle of about 19.16°. In some embodiments, the XRPD pattern of co-crystal Form A may additionally comprise significant peaks at 2θ angle of about 10.74°, about 25.3°, and about 24.07°. In some embodiments, the XRPD pattern of co-crystal Form A may additionally comprise significant peaks at 2θ angle of about 14.54°, about 16.5°, about 13.44°, about 23.42°, and about 8.62°. In some embodiments, the XRPD pattern further comprises a significant peak at a 2θ angle of about 10.74°, about 25.3°, about 24.07°, about 14.54°, about 16.5°, about 13.44°, about 23.42°, or about 8.62°.

In one embodiment, weight loss (1.3%) is observed between 40-200° C. by TGMS for co-crystal Form A.

In one embodiment, co-crystal Form A is characterized by a DSC plot comprising a sharp endothermic event at a temperature of about 255° C.

In some embodiments, co-crystal Form A has ratio of psilocybin to psilocin of about 1:1, about 1.1:1, about 1.2:1, about 1.3:1, about 1.4:1, about 1.5:1, about 1.6:1, about 1.7:1, about 1.8:1, about 1.9:1, about 2:1, about 1:1.1, about 1:1.2, about 1:1.3, about 1:1.4, about 1:1.5, about 1:1.6, about 1:1.7, about 1:1.8, about 1:1.9, or about 1:2. In some embodiments, co-crystal Form A has ratio of psilocybin to psilocin of about 1:1.

In some embodiments, co-crystal Form A has a chemical purity of about 95% or greater, is about 96% or greater, is about 97% or greater, is about 98% or greater, is about 98.5% or greater, is about 99% or greater, is about 99.5% or greater, or is about 99.8% or greater. In some embodiments, co-crystal Form A is substantially pure.

In some embodiments, co-crystal Form A contains not more than about 0.01 mol %, about 0.02 mol %. about 0.03 mol %, about 0.04 mol %, about 0.05 mol %, about 0.06 mol %, about 0.07 mol %, about 0.08 mol %, about 0.09 mol %, about 0.1 mol %, about 0.15 mol %, about 0.2 mol %, about 0.25 mol %, about 0.3 mol %, about 0.35 mol %, about 0.4 mol %, about 0.45 mol %, about 0.5 mol %, about 0.55 mol %, about 0.6 mol %, about 0.65 mol %, about 0.7 mol %, about 0.75 mol %, about 0.8 mol %, about 0.85 mol %, about 0.9 mol %, about 0.95 mol %, about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, of other solid forms, e.g., amorphous form. In some embodiments, co-crystal Form A of is substantially free of other solid forms.

In some embodiments, co-crystal Form A of psilocin and psilocybin comprises about 99 mol % co-crystal Form A, about 0.5 mol % psilocin, and about 0.5 mol % psilocybin; about 98 mol % co-crystal Form A, about 1.0 mol % psilocin, and about 1.0 mol % psilocybin; about 97 mol % co-crystal Form A, about 1.5 mol % psilocin, and about 1.5 mol % psilocybin; about 96 mol % co-crystal Form A, about 2.0 mol % psilocin, and about 2.0 mol % psilocybin; about 95 mol % co-crystal Form A, about 2.5 mol % psilocin, and about 2.5 mol % psilocybin; about 94 mol % co-crystal Form A, about 3.0 mol % psilocin, and about 3.0 mol % psilocybin; about 93 mol % co-crystal Form A, about 3.5 mol % psilocin, and about 3.5 mol % psilocybin; about 92 mol % co-crystal Form A, about 4.0 mol % psilocin, and about 4.0 mol % psilocybin; about 91 mol % co-crystal Form A, about 4.5 mol % psilocin, and about 4.5 mol % psilocybin; or about 90 mol % co-crystal Form A, about 5.0 mol % psilocin, and about 5.0 mol % psilocybin.

In some embodiments, co-crystal Form A is anhydrous.

In some embodiments, co-crystal Form A is a salt formed between psilocin and psilocybin.

In some embodiments, co-crystal Form A is a co-crystal formed between psilocin and psilocybin.

In some embodiments, co-crystal Form A is characterized by unit cell parameters which are substantially equal to the following:

### Unit Cell Dimensions:

a=9.3674(3) A
b=11.2660(6) A
c=24.2741(9) A
α=90 degrees
β=90 degrees
γ=90 degrees
Space group=P2₁2₁2₁
Molecules/asymmetric unit=1

In some embodiments, the unit cell parameters were measured about 296K.

In some embodiments, co-crystal Form A has a geometry of hydrogen bonds substantially as listed in Table A.

TABLE A

| D-H . . . A | D-H [Å] | H . . . A [Å] | D . . . A [Å] | D-H . . . A [°] |
|---|---|---|---|---|
| O1-H1 . . . N23ⁱ | 0.88(2) | 1.60(2) | 2.46(2) | 162(2) |
| N11-H11 . . . O3ⁱⁱ | 0.96(5) | 2.17(4) | 2.97(4) | 140(6) |
| N17-H17 . . . O2ⁱⁱⁱ | 0.85(7) | 1.83(7) | 2.59(6) | 147(8) |
| N28-H28 . . . O1^{iv} | 0.87(7) | 2.32(6) | 3.13(5) | 153(10) |
| O34-H34 . . . O3^{v} | 0.88(10) | 1.69(8) | 2.49(7) | 151(5) |

In some embodiments, co-crystal Form A has atomic coordinates of non-hydrogen atoms substantially as listed in Table B.

TABLE B

| | x | y | z | U(eq) |
|---|---|---|---|---|
| O1 | 0.1251(14) | 0.0326(16) | 0.4198(4) | 0.0253 |
| O2 | 0.077(3) | 0.0094(13) | 0.3153(5) | 0.0253 |
| O3 | 0.190(3) | 0.190(2) | 0.3483(6) | 0.0253 |
| P4 | 0.0916(7) | 0.0915(7) | 0.3636(3) | 0.0253 |
| O5 | −0.054(2) | 0.157(3) | 0.3724(5) | 0.0253 |
| C6 | −0.1818(8) | 0.102(5) | 0.3771(8) | 0.0253 |
| C7 | −0.221(2) | 0.009(6) | 0.4089(13) | 0.0253 |
| C8 | −0.357(3) | −0.030(9) | 0.4084(19) | 0.0253 |
| C9 | −0.466(2) | 0.022(11) | 0.375(2) | 0.0253 |
| C10 | −0.4261(9) | 0.116(10) | 0.3436(15) | 0.0253 |
| N11 | −0.512(3) | 0.188(12) | 0.3068(16) | 0.0253 |
| C12 | −0.386(5) | 0.277(10) | 0.2836(13) | 0.0253 |
| C13 | −0.265(4) | 0.250(7) | 0.3069(10) | 0.0253 |
| C14 | −0.2791(18) | 0.156(7) | 0.3445(10) | 0.0253 |
| C15 | −0.139(6) | 0.313(5) | 0.2890(13) | 0.0253 |
| C16 | −0.082(4) | 0.261(6) | 0.2343(10) | 0.0253 |
| N17 | 0.054(6) | 0.324(5) | 0.2219(15) | 0.0253 |
| C18 | 0.140(5) | 0.246(6) | 0.1809(13) | 0.0253 |
| C19 | 0.146(7) | 0.370(3) | 0.269(2) | 0.0253 |
| C21 | 0.8842(18) | 0.5884(19) | 0.9383(6) | 0.0253 |
| C22 | 0.7490(17) | 0.7295(10) | 0.9827(10) | 0.0253 |
| N23 | 0.8087(12) | 0.6116(8) | 0.9893(5) | 0.0253 |
| C24 | 0.7012(15) | 0.5244(13) | 1.0037(8) | 0.0253 |
| C25 | 0.591(2) | 0.533(3) | 0.9591(11) | 0.0253 |
| C26 | 0.508(4) | 0.422(3) | 0.9490(17) | 0.0253 |
| C27 | 0.543(5) | 0.306(3) | 0.963(2) | 0.0253 |
| N28 | 0.435(7) | 0.236(4) | 0.942(3) | 0.0253 |
| C29 | 0.337(7) | 0.302(6) | 0.917(3) | 0.0253 |
| C30 | 0.212(8) | 0.268(7) | 0.891(3) | 0.0253 |
| C31 | 0.124(7) | 0.356(9) | 0.868(3) | 0.0253 |
| C32 | 0.161(6) | 0.474(8) | 0.872(3) | 0.0253 |
| C33 | 0.292(5) | 0.508(6) | 0.899(3) | 0.0253 |
| O34 | 0.330(4) | 0.628(6) | 0.906(3) | 0.0253 |
| C35 | 0.379(5) | 0.422(5) | 0.920(2) | 0.0253 |

In some embodiments, co-crystal Form A has atomic coordinates of hydrogen atoms substantially as listed in Table C.

TABLE C

| | x | y | z | U(eq) |
|---|---|---|---|---|
| H1 | 0.1396(11) | 0.074(2) | 0.4502(3) | 0.0253 |
| H7 | −0.151(3) | −0.029(5) | 0.4319(14) | 0.0253 |
| H8 | −0.379(5) | −0.097(9) | 0.431(2) | 0.0253 |
| H9 | −0.563(3) | −0.005(13) | 0.374(2) | 0.0253 |
| H11 | −0.612(2) | 0.172(14) | 0.3030(19) | 0.0253 |
| H12 | −0.389(6) | 0.337(10) | 0.2556(15) | 0.0253 |
| H15A | −0.075(5) | 0.294(4) | 0.3185(12) | 0.0253 |
| H15B | −0.147(8) | 0.398(5) | 0.2858(18) | 0.0253 |
| H16A | −0.146(5) | 0.280(7) | 0.2048(11) | 0.0253 |
| H16B | −0.074(3) | 0.176(5) | 0.2375(8) | 0.0253 |
| H17 | 0.041(8) | 0.386(5) | 0.2026(19) | 0.0253 |
| H18A | 0.205(4) | 0.181(6) | 0.1844(12) | 0.0253 |
| H18B | 0.203(6) | 0.295(7) | 0.1601(17) | 0.0253 |
| H18C | 0.075(4) | 0.206(7) | 0.1563(10) | 0.0253 |
| H19A | 0.213(7) | 0.336(3) | 0.2941(19) | 0.0253 |
| H19B | 0.086(8) | 0.405(2) | 0.296(2) | 0.0253 |
| H19C | 0.211(9) | 0.429(4) | 0.255(2) | 0.0253 |
| H21A | 0.951(2) | 0.627(3) | 0.9141(7) | 0.0253 |
| H21B | 0.817(3) | 0.575(3) | 0.9093(5) | 0.0253 |
| H21C | 0.944(2) | 0.520(2) | 0.9427(10) | 0.0253 |
| H22A | 0.778(2) | 0.8072(10) | 0.9708(14) | 0.0253 |
| H22B | 0.715(2) | 0.7579(14) | 1.0176(12) | 0.0253 |
| H22C | 0.6710(17) | 0.7266(16) | 0.9571(11) | 0.0253 |
| H24A | 0.6599(17) | 0.542(2) | 1.0390(8) | 0.0253 |
| H24B | 0.742(2) | 0.4463(11) | 1.0054(11) | 0.0253 |
| H25A | 0.635(3) | 0.565(3) | 0.9267(9) | 0.0253 |
| H25B | 0.5179(14) | 0.587(3) | 0.9706(16) | 0.0253 |
| H27 | 0.629(5) | 0.2905(18) | 0.984(3) | 0.0253 |
| H28 | 0.436(8) | 0.159(4) | 0.946(4) | 0.0253 |
| H30 | 0.186(9) | 0.185(8) | 0.890(4) | 0.0253 |
| H31 | 0.037(8) | 0.334(10) | 0.851(4) | 0.0253 |
| H32 | 0.100(6) | 0.533(9) | 0.856(4) | 0.0253 |
| H34 | 0.294(4) | 0.682(7) | 0.884(3) | 0.0253 |

In some embodiments, the crystalline form of psilocin and psilocybin is co-crystal Form B.

In some embodiments, co-crystal Form B is characterized by a XRPD pattern comprising a significant peak at a 2θ angle of about 18.54°. In some embodiments, co-crystal Form B is characterized by a XRPD pattern of co-crystal Form B may additionally comprise a significant peak at 2θ angle of about 8.54°. In some embodiments, co-crystal Form B is characterized by a XRPD pattern of co-crystal Form B may additionally comprise significant peaks at 2θ angle of about 22.78°, about 14.27°, and about 21.12°. In some embodiments, co-crystal Form B is characterized by a XRPD pattern of co-crystal Form B may additionally comprise significant peaks at 2θ angle of about 14.12°, about 10.05°, about 9.94°, about 24.94°, and about 25.02°.

In one embodiment, weight loss (15.8%) is observed between 40-200° C. by TGMS for co-crystal Form B.

In one embodiment, co-crystal Form B is characterized by a DSC plot comprising a broad endothermic event at a temperature of about 168.2° C.

In some embodiments, co-crystal Form B has ratio of psilocybin to psilocin of about 1:1, about 1.1:1, about 1.2:1, about 1.3:1, about 1.4:1, about 1.5:1, about 1.6:1, about 1.7:1, about 1.8:1, about 1.9:1, about 2:1, about 1:1.1, about 1:1.2, about 1:1.3, about 1:1.4, about 1:1.5, about 1:1.6, about 1:1.7, about 1:1.8, about 1:1.9, or about 1:2. In some embodiments, co-crystal Form B has ratio of psilocybin to psilocin of about 1.3:1. In some embodiments, co-crystal Form B has ratio of psilocybin to psilocin of about 1:1.

In some embodiments, co-crystal Form B has a chemical purity of about 95% or greater, is about 96% or greater, is about 97% or greater, is about 98% or greater, is about 98.5% or greater, is about 99% or greater, is about 99.5% or greater, or is about 99.8% or greater. In some embodiments, co-crystal Form B is substantially pure.

In some embodiments, co-crystal Form B contains not more than about 0.01 mol %, about 0.02 mol %. about 0.03 mol %, about 0.04 mol %, about 0.05 mol %, about 0.06 mol %, about 0.07 mol %, about 0.08 mol %, about 0.09 mol %, about 0.1 mol %, about 0.15 mol %, about 0.2 mol %, about 0.25 mol %, about 0.3 mol %, about 0.35 mol %, about 0.4 mol %, about 0.45 mol %, about 0.5 mol %, about 0.55 mol %, about 0.6 mol %, about 0.65 mol %, about 0.7 mol %, about 0.75 mol %, about 0.8 mol %, about 0.85 mol %, about 0.9 mol %, about 0.95 mol %, about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, of other solid forms, e.g., amorphous form. In some embodiments, co-crystal Form B of is substantially free of other solid forms.

In some embodiments, co-crystal Form B is anhydrous.

In some embodiments, co-crystal Form B is a salt formed between psilocin and psilocybin.

In some embodiments, co-crystal Form B is a co-crystal formed between psilocin and psilocybin.

In some embodiments, the crystalline form psilocin and psilocybin is co-crystal Form C.

In some embodiments, co-crystal Form C is characterized by a XRPD pattern comprising a significant peak at a 2θ angle of about 9.18°. In some embodiments, co-crystal Form C is characterized by a XRPD pattern of co-crystal Form C may additionally comprise a significant peak at 2θ angle of about 17.95°. In some embodiments, co-crystal Form C is characterized by a XRPD pattern of co-crystal Form C may additionally comprise significant peaks at 2θ angle of about 10.42°, about 24.22°, and about 18.38°. In some embodiments, co-crystal Form C is characterized by a XRPD pattern of co-crystal Form C may additionally comprise significant peaks at 2θ angle of about 19.82°, about 17.46°, about 14.82°, about 22.38°, and about 14.06°.

In one embodiment, weight loss (13.5%) is observed between 40-160° C. by TGMS for co-crystal Form C.

In one embodiment, co-crystal Form C is characterized by a DSC plot comprising a broad endothermic event at a temperature of about 25-140° C., with a peak at 98.7° C.

In some embodiments, co-crystal Form C has ratio of psilocybin to psilocin of about 1:1, about 1.1:1, about 1.2:1, about 1.3:1, about 1.4:1, about 1.5:1, about 1.6:1, about 1.7:1, about 1.8:1, about 1.9:1, about 2:1, about 1:1.1, about 1:1.2, about 1:1.3, about 1:1.4, about 1:1.5, about 1:1.6, about 1:1.7, about 1:1.8, about 1:1.9, or about 1:2. In some embodiments, co-crystal Form C has ratio of psilocybin to psilocin of about 1:1.

In some embodiments, co-crystal Form C has a chemical purity of about 95% or greater, is about 96% or greater, is about 97% or greater, is about 98% or greater, is about 98.5% or greater, is about 99% or greater, is about 99.5% or greater, or is about 99.8% or greater. In some embodiments, co-crystal Form C is substantially pure.

In some embodiments, co-crystal Form C contains not more than about 0.01 mol %, about 0.02 mol %. about 0.03 mol %, about 0.04 mol %, about 0.05 mol %, about 0.06 mol %, about 0.07 mol %, about 0.08 mol %, about 0.09 mol %, about 0.1 mol %, about 0.15 mol %, about 0.2 mol %, about 0.25 mol %, about 0.3 mol %, about 0.35 mol %, about 0.4 mol %, about 0.45 mol %, about 0.5 mol %, about 0.55 mol %, about 0.6 mol %, about 0.65 mol %, about 0.7 mol %, about 0.75 mol %, about 0.8 mol %, about 0.85 mol %, about 0.9 mol %, about 0.95 mol %, about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, of other solid forms, e.g., amorphous form. In some embodiments, co-crystal Form C of is substantially free of other solid forms.

In some embodiments, co-crystal Form C is anhydrous.

In some embodiments, co-crystal Form C is a salt formed between psilocin and psilocybin.

In some embodiments, co-crystal Form C is a co-crystal formed between psilocin and psilocybin.

### Pharmaceutical Compositions

In one embodiment, the present application provides pharmaceutical composition comprising a crystalline form of psilocin and psilocybin and a pharmaceutically acceptable excipient.

In one embodiment the crystalline form is co-crystal Form A, co-crystal Form B or Form C. In one embodiment, co-crystal Form A is characterized by a XRPD pattern comprising a significant peak at a 2θ angle of about 10.1°; the XRPD pattern further comprises a significant peak at a 2θ angle of about 19.16°; at a 2θ angle of about 10.74°, about 25.3°, and about 24.07°; and/or the XRPD pattern further comprises a significant peak at a 2θ angle of about 14.54°, about 16.5°, about 13.44°, about 23.42°, and about 8.62°. In one embodiment, the XRPD pattern further comprises a significant peak at a 2θ angle of about 10.74°, about 25.3°, about 24.07°, about 14.54°, about 16.5°, about 13.44°, about 23.42°, or about 8.62°. In one embodiment, co-crystal Form A is characterized by a DSC plot comprising a sharp endothermic event at a temperature of about 255° C. In one embodiment, co-crystal Form A has a ratio of psilocybin to psilocin of about 1:1. In one embodiment, co-crystal Form A has a chemical purity of about 95% or greater. In some embodiments, the pharmaceutical composition of co-crystal Form A contains not more than about 5 mol % of other solid forms. In some embodiments, the pharmaceutical composition of co-crystal Form A of psilocin and psilocybin comprises about 95 mol % co-crystal Form A, about 2.5 mol % psilocin, and about 2.5 mol % psilocybin. In one embodiment, co-crystal Form A is a co-crystal formed between psilocin and psilocybin. In one embodiment, co-crystal Form A is a salt formed between psilocin and psilocybin.

In one embodiment, co-crystal Form A is characterized by unit cell parameters which are substantially equal to the following:

### Unit Cell Dimensions:

a=9.3674(3) A
b=11.2660(6) A
c=24.2741(9) A
α=90 degrees
β=90 degrees
γ=90 degrees
Space group=P2₁2₁2₁
Molecules/asymmetric unit=1

In one embodiment, the unit cell parameters were measured about 296K. In one embodiment, co-crystal Form A has a geometry of hydrogen bonds substantially as listed in Table A. In one embodiment, co-crystal Form A has atomic coordinates of non-hydrogen atoms substantially as listed in Table B. In one embodiment, co-crystal Form A has atomic coordinates of hydrogen atoms substantially as listed in Table C. In one embodiment, co-crystal Form A is a co-crystal formed between psilocin and psilocybin. In one embodiment, co-crystal Form A is a salt formed between psilocin and psilocybin.

In one embodiment, co-crystal Form B is characterized by a XRPD pattern comprising a significant peak at a 2θ angle of about 18.54°; the XRPD pattern further comprises a significant peak at a 2θ angle of about 8.54°; the XRPD pattern further comprises a significant peak at a 2θ angle of about 22.78°, about 14.27°, and about 21.120 and/or the XRPD pattern further comprises a significant peak at a 2θ angle of about 14.12°, about 10.05°, about 9.94°, about 24.94°, and about 25.02°. In one embodiment, co-crystal Form B is characterized by a DSC plot comprising a broad endothermic event at a temperature of about 168.2° C. In one embodiment, co-crystal Form B has ratio of psilocybin to psilocin of about 1.3:1. In one embodiment, co-crystal Form B has ratio of psilocybin to psilocin of about 1:1. In one embodiment, co-crystal Form B has a chemical purity of about 95% or greater. In some embodiments, the pharmaceutical composition of co-crystal Form B contains not more than about 5 mol % of other solid forms. In some embodiments, the pharmaceutical composition of co-crystal Form B of psilocin and psilocybin comprises about 95 mol % co-crystal Form B, about 2.5 mol % psilocin, and about 2.5 mol % psilocybin. In one embodiment, co-crystal Form B is a co-crystal formed between psilocin and psilocybin. In one embodiment, co-crystal Form B is a salt formed between psilocin and psilocybin.

In one embodiment, co-crystal Form C is characterized by a XRPD pattern comprising a significant peak at a 2θ angle of about 9.18°; the XRPD pattern further comprises a significant peak at a 2θ angle of about 17.95°; the XRPD pattern further comprises a significant peak at a 2θ angle of about 10.42°, about 24.22°, and about 18.38°; and/or the XRPD pattern further comprises a significant peak at a 2θ angle of about 19.82°, about 17.46°, about 14.82°, about 22.38°, and about 14.06°. In one embodiment, co-crystal Form C is characterized by a DSC plot comprising a broad endothermic event at a temperature of about 25-140° C., with a peak at 98.7° C. In one embodiment, co-crystal Form C has ratio of psilocybin to psilocin of about 1:1. In one embodiment, co-crystal Form C has a chemical purity of about 95% or greater. In some embodiments, the pharmaceutical composition of co-crystal Form C contains not more than about 5 mol % of other solid forms. In some embodiments, the pharmaceutical composition of co-crystal Form C of psilocin and psilocybin comprises about 95 mol % co-crystal Form C, about 2.5 mol % psilocin, and about 2.5 mol % psilocybin. In one embodiment, co-crystal Form C is a co-crystal formed between psilocin and psilocybin. In one embodiment, co-crystal Form C is a salt formed between psilocin and psilocybin.

As used herein, “pharmaceutical composition” refers to a formulation comprising an active ingredient, and optionally a pharmaceutically acceptable carrier, diluent or excipient. The term “active ingredient” can interchangeably refer to an “effective ingredient”, and is meant to refer to any agent that is capable of inducing a sought-after effect upon administration. Examples of active ingredient include, but are not limited to, chemical compound, drug, therapeutic agent, small molecule, etc. In one embodiment, the active ingredient is a crystalline form of psilocybin and psilocin, such as co-crystal Form A, co-crystal Form B or co-crystal Form C described herein.

The terms “excipient” and “pharmaceutically acceptable excipient” as used herein are intended to be generally synonymous, and is used interchangeably with, the terms “carrier,” “pharmaceutically acceptable carrier,” “diluent,” “pharmaceutically acceptable diluent.” By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

In one embodiment, the pharmaceutically acceptable excipient is phosphate buffer; citrate buffer; ascorbic acid; methionine; octadecyldimethylbenzyl ammonium chloride; hexamethonium chloride; benzalkonium chloride; benzethonium chloride; phenol alcohol; butyl alcohol; benzyl alcohol; methyl paraben; propyl paraben; catechol; resorcinol; cyclohexanol; 3-pentanol; m-cresol; low molecular weight (less than about 10 residues) polypeptides; serum albumin; gelatin; immunoglobulins; polyvinylpyrrolidone glycine; glutamine; asparagine; histidine; arginine; lysine; monosaccharides; disaccharides; glucose; mannose; dextrins; EDTA; trehalose, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose, including eutectic solvents, eutectic-based ionic liquids, ionic liquids, sorbitol; sodium; saline; metal surfactants; non-ionic surfactants; polyethylene glycol (PEG); magnesium stearate; water; alcohol; saline solution; glycol; mineral oil, dimethyl sulfoxide (DMSO) or a combination thereof.

The active compound can be effective over a wide dosage range and can be generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

### Ca²⁺ Release Assay

Cell Culture—AequoScreen™ Chinese hamster ovary (CHO)-K1 cells stably expressing human 5-HT2A or 5-HT2B (h5HT2A, h5HT2B) were maintained at 37° C., 5% CO₂ in F-12 DMEM containing 1 mM L-glutamine, 10% FBS, and 1% Pen/Strep.

Ca²⁺ Assay—Release of Ca²⁺ into the cellular cytosol upon GPCR-dependent activation of Ga_{q/11} was determined using the AequoScreen™ assay in CHO-K1 cells stably expressing h5HT2A or h5HT2B. In addition to the receptor of choice, the AequoZen cell lines stably express the photoprotein apoaequorin, which possesses three Ca²⁺ binding sites. Upon binding Ca²⁺, apoaequorin becomes activated to aequorin and converts coelenterazine to coelenteramide, CO₂, and light. Light production is therefore directly proportional to the release of Ca²⁺ from intracellular stores upon GPCR activation. Cells were seeded into flat bottom, white bottom, white wall, poly-D-lysine coated 96-well plates (20,000 cells/well) and incubated overnight in Opti-MEM containing 1% FBS at 37° C. and 5% CO₂. Following this, Opti-MEM media was removed and replaced with fresh Opti-MEM containing 5 μM coelenterazine h (Promega, Madison, WI) and 1% FBS (final volume 50 μL) and cells were incubated at 25° C. for 4 h. After these 4 h, cells were treated with 50 μL 2× concentrated ligand solutions to achieve the concentrations indicated and centrifuged at 1,000×g for 1 min at room temperature. Chemiluminescence was then immediately measured on a Cytation5 plate reader (top read, gain 200, integration time 10,000 msec). All experiments included a vehicle control (5% DMSO) and LSD was included in all experiments as a reference agonist.

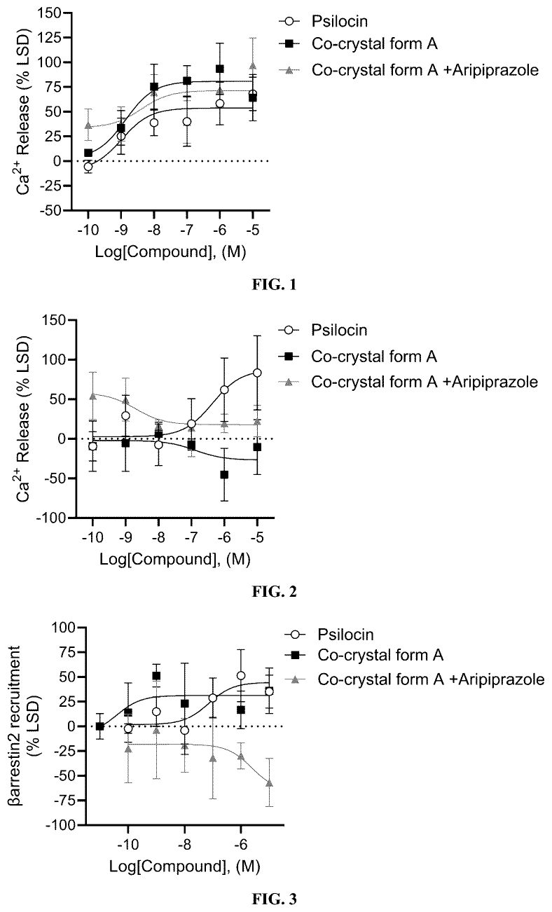

Results are shown in FIGS. 1 and 2. FIG. 1 shows that calcium release (as a percentage of LSD) at 5-HT2A receptors for co-crystal form A is similar to that of psilocin. Surprisingly, however, when combining both co-crystal form A with aripiprazole (a 5-HT2 receptor antagonist), there is no decrease in calcium release (as a percentage of LSD) at the 5-HT2A receptor in this model. FIG. 2 shows that, in contrast to the findings at the 5-HT2A receptor, at the 5-HT2B receptor calcium release (as a percentage of LSD) for co-crystal form A is lower than that of psilocin at the highest doses. This is also the case following administration of the combination of co-crystal form A with aripiprazole (a 5-HT2B receptor antagonist), where these is again a reduction in calcium release (as a percentage of LSD) at higher doses when compared to psilocin.

### Example 2

βarrestin2 Recruitment Assay

Cell Culture—CHO-K1 cells were transfected with either NanoBit™ smBiT h5HT2A or h5HT2B plasmids and lgBiT βarrestin2 plasmid. When h5HT2A or 2B and βarrestin2 come into contact with one another the smBiT and lgBiT peptides complement each other to produce a complete NanoBit™ luciferase enzyme. When complete, the enzyme is able to catalyze the breakdown of the molecule coelenterazine to produce light. Emitted light levels are proportional to interactions between receptor and βarrestin2. Cells were maintained at 37° C., 5% CO₂ in F-12 DMEM containing 1 mM L-glutamine, 10% FBS, and 1% Pen/Strep. For transfections, CHO-K1 cells were seeded in 6-well plates (500,000 cells per well) and incubated overnight in standard media as described above. Following overnight incubation, the cells were transfected with 1 g of the h5HT2A or h5HT2B and the βarrestin2 NanoBiT plasmids. Transfections were carried out using Lipofectamine 3000 (Fisher Scientific, Ottawa, ON), according to the manufacturer's protocol in Opti-MEM media containing 1% FBS. After 24 h, the cells were seeded in poly-D-lysine coated 96-well plates at a density of 20,000 cells per well and incubated overnight maintained at 37° C., 5% CO₂ in F-12 DMEM containing 1 mM L-glutamine, 10% FBS, and 1% Pen/Strep prior to use.

βarrestin2 assay-After seeding at 20,000 cells/well in flat bottom, white bottom, white wall, poly-D-lysine coated 96-well plates, transfected CHO-K1 cells were rinsed twice with Hank's buffered salt solution (HBSS), and 100 μL of HBSS was added to each of the wells. To this, 25 μL of Nano-Glo™ Live Cell Substrate was added (diluted 1/20 in the Nano-Glo LCS Dilution buffer, according to the manufacturer's protocol), and the plate was transferred to a Cytation5 plate reader. After a 1 h equilibration phase, 10 L of the 13.5× concentrated ligand solutions were added, and the luminescent signal was monitored continuously for 2 h (top read, gain 200, integration time 10,000 msec). Luminescent data for the first 30 min were used to automatically generate a time-by-luminescence area under the curve (AUC) for each concentration of compound. AUC data were then used for subsequent analyses. All experiments included a vehicle control (5% DMSO) and lysergic acid diethylamide (LSD) was included in all experiments as a reference agonist.

Results are shown in FIG. 3. FIG. 3 shows that βarrestin2 recruitment (as a percentage of LSD) at 5-HT2A receptors for co-crystal form A is similar to that of psilocin, as found with calcium release shown FIG. 1. In contrast to calcium release at the 5-HT2A receptor, shown in FIG. 1, when administering co-crystal form A with aripiprazole there is a significant decrease in βarrestin2 recruitment (as a percentage of LSD) at the 5-HT2A receptor in this model, most marked at the highest doses. This suggests an unexpected interaction of these two compounds at this receptor to have a combined impact on βarrestin2 recruitment.

### Example 3

### BDNF Expression, Neuronal Growth, and Neuronal Proliferation

Human Cortical Brain Organoid Culturing Conditions. Human induced pluripotent stem cells (hiPSCs) were cultured in 6-well tissue culture plates coated with Matrigel. First, the cells were incubated with DMEM/F-12 media containing Dispase for 7 minutes, followed by two washes of DMEM/F-12. Cells were scraped, centrifuged, and the pellet was resuspended in mTESR1 medium. Resuspended hiPSCs were re-plated and allowed to grow in mTESR1 for 48 hours. Embryoid bodies (EB) were generated by dissociating hiPSCs and incubating with Accutase for 1 minute prior to transfer to a conical tube for centrifugation in DMEM/F-12 medium. The cell suspension was diluted to a concentration of 60,000 live cells/ml and Y-27632 and 5% (v/v) heat-inactivated FBS was added. 150 μL of cell suspension was added to U-bottom ultra-low-attachment 96-well plates and placed at 37° C. On day 2, neural induction medium was added to each well. On days 4, 6, and 8, fresh neural induction media was added. On day 10˜8 EBs were transferred to each well in an ultra-low-attachment 6-well plate. Neural differentiation medium was added to each well and they were placed on an orbital shaker inside a 37C incubator at 80 rpm. On days 12, 14, and 16, medium was aspirated, and fresh neural differentiation medium added. On day 18, neural differentiation medium was added to each well. Neural differentiation medium was then changed every 4 days until assay commencement.

Mouse Breeding & Drug Injection Details. 14-week-old female CD1 mice, with an average weight of 23 g, were injected intraperitoneally once per day for 14 consecutive days with the following drug treatments: 1) vehicle (230 L), 2) Co-crystal Form A (0.3 mg/kg), 3) Aripiprazole (0.3 mg/kg), and 4) Co-crystal Form A+Aripiprazole (0.3 mg/kg). A solution of 0.67% DMSO in PBS was used as the vehicle. For immunohistochemistry sampling, EdU (5-Ethynyl-2′-deoxyuridine, Lumiprobe, 100 μg/g) was injected together with the drugs for the first 7 days. On the day following the 14-day treatment period, the brains of the mice were dissected for subsequent analyses, including immunohistochemistry and electrophysiology (E-phys).

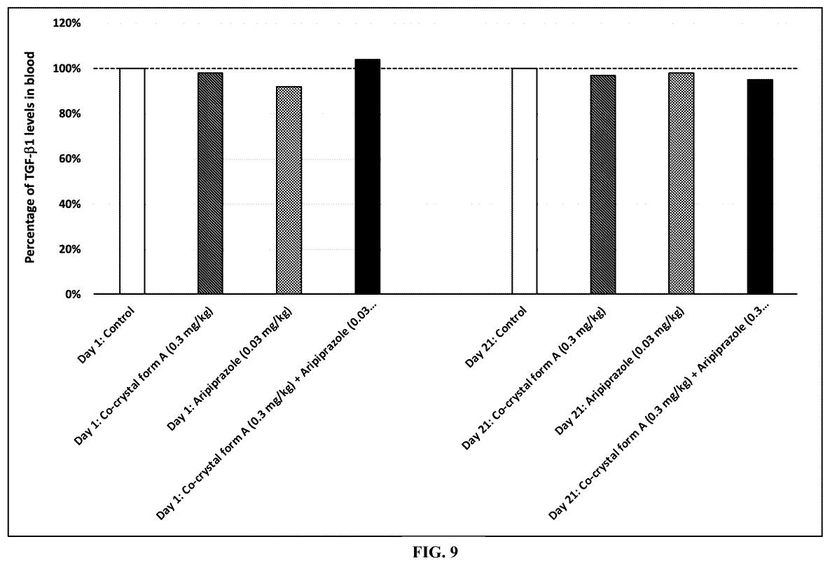

Quantitative Polymerase Chain Reaction (qPCR). To measure BDNF expression Trizol was used to extract RNA from the organoids. Initially, one organoid was placed in a 1.5 mL tube and 250 μL of Trizol was added and the tissue was homogenized with a plastic stirrer. 62.5 μL of Choloroform was added and the tube was inverted and left at room temperature for 2 minutes and then centrifuged at 12,000×g for 15 minutes at 4° C. Post-centrifugation, the aqueous middle layer was carefully transferred to a fresh 1.5 ml tube, and 1:1 2-propanol was added. The tube was again centrifuged at 12,000×g for 20 minutes, the supernatant was then poured off and the tube was left to dry. 1000 μL of 70% ethanol was added and the tube was repetitively inverted to mix and was then centrifuged at 4° C. for 5 minutes at 12,000×g, the supernatant was then poured off. Following 15 minutes of air drying, the pellet was resuspended in 20 μL of sterile water and concentration determined using a Nanodrop machine. Reverse transcription was completed with 1 μg of RNA, 2 μL gDNA wipeout buffer 7×, and the solution was brought to a total volume of 14 μL with the addition of RNA free water. Tubes were incubated at 42° C. for 2 minutes and then placed on ice. Each tube then received 1 μL of Reverse Transcriptase (RT), 4 μL of RT buffer, 1 μl of RT primer mix, to bring the total volume to 20 μl mastermix per replicate. Tubes were then incubated at 42° C. for 15 minutes and 3 minutes at 95° C. Qiagen SYBR Green kit was used for qPCR, each well on a 384 well plate received 5 μL SYBR Green, 2.8 μL RNase free water, 0.6 μL of each the forward and reverse primer (housekeeping were Y-WHAZ & GAPDH, target was BDNF), and 1 μl of cDNA. Results are shown in FIGS. 8 and 9.

Brain Organoid Neurite Outgrowth Assay. 6-month-old cortical organoids (CO) & median ganglion eminence organoids (MGEO) were fused to become an assembloid and were treated for 10 days with the following drug conditions: 1) vehicle, 2) Co-crystal Form A (500 nM), 3) Aripiprazole (500 nM), and 4) Co-crystal Form A+Aripiprazole (500 nM). A solution of 0.1% DMSO in growth media was used as the vehicle. The media were replaced daily with fresh media containing the drugs. After the 10-day drug treatment, 20 mm×20 mm square cover glasses on each well of a 6-well plate were coated with 1% Geltrex in DMEM/F12+Glutamax for 2 hours at room temperature. The coated plates were then washed with 2 ml of growth media, and 3 mL of warmed fresh growth media was added to each well. Following plate preparation, the drug-treated organoids were sliced 9 times using surgical blades and transferred onto the Geltrex-coated cover glasses in the 6-well plates. The plates were gently swirled to evenly distribute the organoid fragments. The organoid fragments were incubated undisturbed for 3 days to allow attachment and neurite growth. After 3 days of incubation, the attached organoid fragments were washed twice with 3 ml of PBS and fixed with 3 ml of 4% paraformaldehyde in PBS for 1 hour at room temperature. The fixed neurite outgrowth samples were washed three times with 3 ml of PBS and then permeabilized with 3 ml of 0.1% Triton X-100 in PBS (PBT) for 10 minutes. The permeabilized samples were subsequently subjected to immunostaining.

For immunostaining, the samples were blocked with 5% normal donkey serum for 1 hour. After blocking, anti-Tuj1 mouse antibody (1:500) in 1 ml of blocking solution was applied overnight at room temperature. Following primary antibody incubation, the samples were washed three times with PBST (0.1% Triton X-100 in PBS). Donkey anti-mouse 488 secondary antibodies (1:500) were then applied for 2 hours at room temperature. After the secondary antibody incubation, the samples were washed three times with PBST and stained with Hoechst 33342 (1:1000 in PBST) for nuclear visualization. After one final wash with PBST, the cover glasses were retrieved and mounted on slide glasses using Aqua-Poly/Mount. Neurite images were obtained using a Zeiss Imager.M2 microscope, and the analysis was performed using Fiji (ImageJ 1.54f) software. Results are shown in FIGS. 10, 11, and 14A-C.

Immunohistochemistry (IHC) & Image Analysis. Sectioned organoids were washed with 1×PBS for 10 minutes on a shaker plate (set to 100), followed by a 0.1% PBT wash. Slides that were not being analyzed for EdU received 1 mL 5% Donkey Serum (diluted in 0.1% PBT) and were incubated at RT for 1 hour. EdU slides (proliferation assay) each received 2× washes of 500 μL of 3% BSA (diluted in PBS) for 2 minutes, then 1 mL of 0.5% triton 100× (diluted in PBS) and were incubated for 20 minutes. Click-iT Plus kit was used for EdU staining and manufacturer instructions were followed. Slides were then washed with 500 μl of 3% BSA (diluted in PBS) and blocked with 5% donkey serum (NDS) for 1 hour. Post-blocking primary antibodies, Ki67 (1:250 in 5% NDS) to EdU slides and both Pax6 (1:250 in 5% NDS) and Synapsin (1:250 in 5% NDS) for non-EdU, were added each with a volume of 200 μL per slide, covered with parafilm, and placed in the 4° C. deli cooler overnight. On day 2, slides were rinsed (4×) with 0.1% PBT for 10 minutes. Rabbit 488 (Rb488) (1:200) was added to label EdU positive cells and Mouse 594 (M594) (1:200) was added to label Ki67 on proliferation assay slides and to label Pax6 and Synapsin on their respective slides. Slides were covered with parafilm and incubated at RT for 2 hours. Slides were rinsed (3×) with 0.1% PBT and then incubated with DAPI (1:1000 in PBS) for 10 minutes at RT. Slides were rinsed (2×) with PBS and coverslips were added. This IHC protocol was used for both mouse and organoid models. Images were gathered via a brightfield microscope, whereby the organoid proliferation, Pax6, and synapsin assays were imaged at 20× and the mouse Pax6 and synapsin were imaged at 63×. Images were obtained on a Zeiss Imager.M2 microscope and ImageJ (Fiji) software was utilized to process the images and count cells based on predetermined fixed parameters set for each model system and counts were graphed using Prism.

Cognitive performance, the Y-maze. Mice will be acclimated to a Y-maze having two familiar arms and one unfamiliar arm 1 day prior to testing (i.e., day-1). One the days of testing mice will be placed in the Y-maze and allowed to freely explore for 5 minutes. Behaviours scored will include total locomotion, time spent in each arm, number of arm entries, and correct alternations between the novel arm and familiar arms. A pro-cognitive effect will be reflected by increased time spent in the novel arm and increased percent correct alternations.

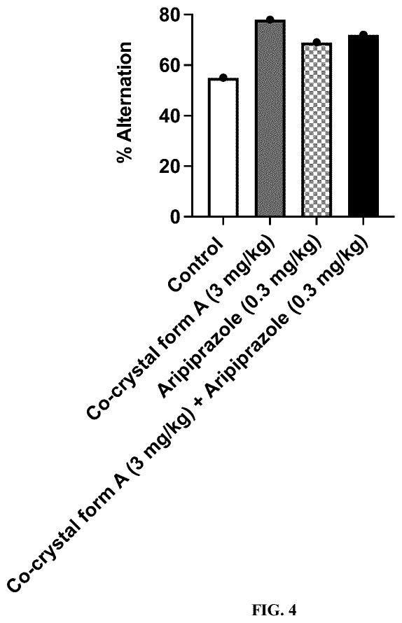

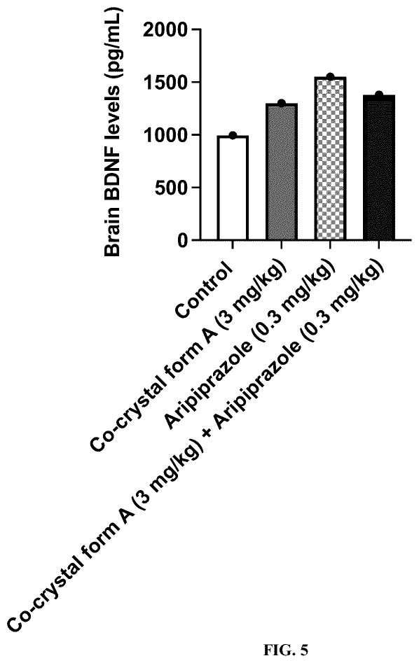

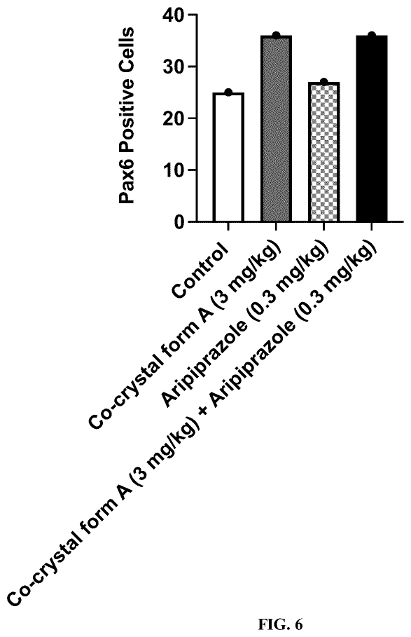

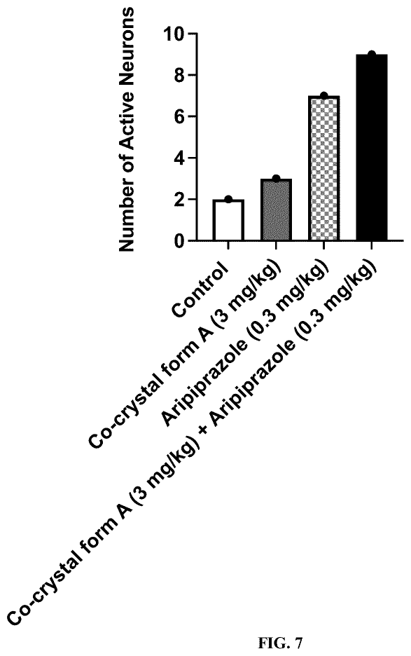

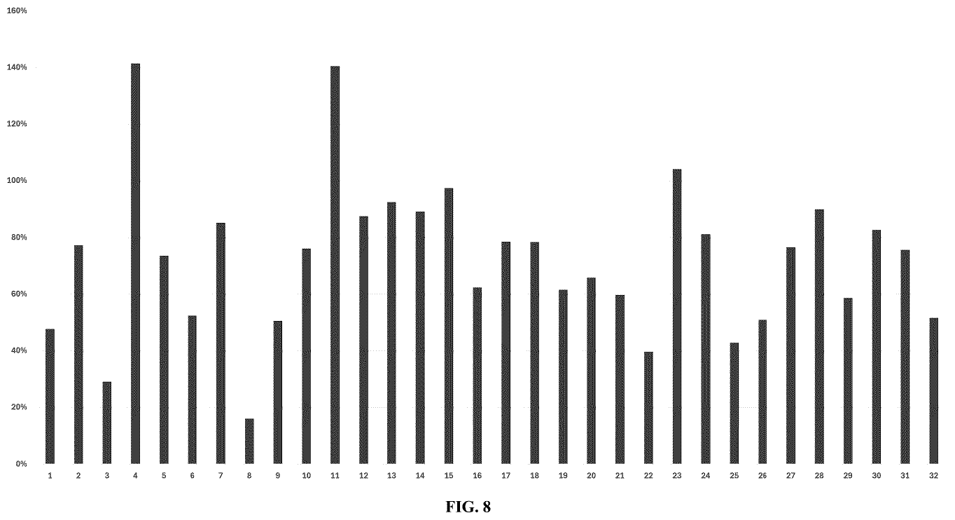

Results are shown in FIGS. 4-7. FIG. 4 shows that, compared to control, Co-crystal Form A had a significant increase in short-term and working memory, Aripiprazole alone also had a smaller but significant increase as well, and that the combination of Co-crystal Form A and Apripiprazole had a significant increase intermediate between the two. FIG. 5 shows that 1 hour after oral administration Co-crystal Form A (3 mg/kg) had a significant increase in brain levels of BDNF, as did Aripiprazole (0.3 mg/kg), and the combination of Co-crystal Form A (3 mg/kg) with Aripiprazole (0.3 mg/kg). FIG. 6 shows the increase in neuronal progenitor/stem cell levels in mice brain following 14-days of oral co-crystal form A (3 mg/kg) alone, and there was an equally strong effect when co-crystal form A (3 mg/kg) was given in combination with aripiprazole (0.3 mg/kg). In contrast, aripiprazole alone (0.3 mg/kg) had no significant effect. FIG. 7 measures the change in neuronal activation in mice after 21-days of oral administration. Co-crystal form A (3 mg/kg) given alone has no significant effect on the number of active neurons. In contrast, aripiprazole (0.3 mg/kg) given alone leads to a significant increase. The combination of both co-crystal form A (3 mg/kg) and aripiprazole (0.3 mg/kg) leads to the greatest increase, suggesting a possible unexpected synergistic effect.

### Example 4

### Reduction in Neuroinflammation and TGF-β Level Quantification

Animals—Adult male and female C57BL/6 mice aged 8-10 weeks were treated with vehicle (saline), 3 mg/kg Co-crystal form A+Aripiprazole, 3 mg/kg psilocybin, 1 mg/kg TrKB inhibitor ANA-12, or a combination of 3 mg/kg Co-crystal form A+Aripiprazole and 1 mg/kg ANA-12 via oral gavage (p.o.) acutely with blood plasma and brain tissue being collected 1 h post-administration. Alternatively, C57BL/6 mice were treated with vehicle (saline), 0.03 mg/kg Co-crystal form A+Aripiprazole, 0.3 mg/kg Co-Crystal form A+Aripiprazole once daily for 21 days via oral gavage and blood plasma and brain tissue being collected 1 h after compound administration on the final (i.e., 21^{st} day).

Cytokine Multiplex Discovery Assay—Tissues were immediately frozen and stored at −80° C. prior to shipping. Blood plasma and brain tissue were analyzed with either (1) the Multiplex Analysis of Cytokines Mouse Cytokine 32-Plex Discovery Assay® with plasma or tissue homogenate; or (2) multi-species tumor growth factor (TGF) β 3-plex Discovery Assay® with plasma or tissue homogenate.

Mouse Cytokine 32-Plex Discovery Assay®: This study used Luminex xMAP technology for multiplexed quantification of 32 Mouse cytokines, chemokines, and growth factors. The multiplexing analysis was performed using the Luminex™ 200 system. Thirty-two markers were simultaneously measured in the samples using Eve Technologies'Mouse Cytokine 32-Plex Discovery Assay® according to the manufacturer's protocol. The 32-plex consisted of Eotaxin, G-CSF, GM-CSF, IFNγ, IL-1a, IL-10, IL-2, IL-3, IL-4, IL-5, IL-6, IL-7, IL-9, IL-10, IL-12(p40), IL-12(p70), IL-13, IL-15, IL-17, IP-10, KC, LIF, LIX, MCP-1, M-CSF, MIG, MIP-1α, MIP-1β, MIP-2, RANTES, TNFα, and VEGF. Assay sensitivities of these markers range from 0.3-30.6 μg/mL for the 32-plex. Individual analyte sensitivity values are available through the MilliporeSigma MILLIPLEX® MAP protocol. Data are presented as either percentage relative to the mean vehicle control or as tissue concentrations (pg/mL).

TGF-β3-Plex Discovery Assay®: This study used Luminex xMAP technology for multiplexed quantification of 3 cytokines: TGF-β1, β2, and β3 in mouse. The multiplexing analysis was performed using the Luminex™ 200 system. Three markers were simultaneously measured in the samples using Eve Technologies' TFG-β 3-Plex Discovery Assay® according to the manufacturer's protocol. The 3-plex consisted of TGF-β1, TGF-β2, and TGF-β3. Assay sensitivities of these markers range from 2.2-6.6 μg/mL for the 3-plex. Individual analyte sensitivity values are available in the MilliporeSigma MILLIPLEX® MAP protocol. Data are presented as either percentage relative to the mean vehicle control or as tissue concentrations (pg/mL).

Results for cytokine biomarkers of neuroinflammation are shown in FIG. 8 with the x-axis numbers represent the following: 1=Eotaxin, 2=G-CSF, 3=GM-CSF, 4=IFNγ, 5=IL-1a, 6=IL-1p, 7=IL-2, 8=IL-3, 9=IL-4, 10=IL-5, 11=IL-6, 12=IL-7, 13=IL-9, 14=IL-10, 15=IL-12p40, 16=IL-12p70, 17=IL-13, 18=IL-15, 19=IL-17, 20=IP-10, 21=KC, 22=LIF, 23=LIX, 24=M-CSF, 25=MCP-1, 26=MIG, 27=MIP-lca, 28=MIP-1, 29=MIP-2, 30=RANTES, 31=TNFα, and 32=VEGF. FIG. 8 shows the percentage change for 32 cytokine biomarkers of neuroinflammation 60 minutes after oral administration of co-crystal form A (3 mg/kg) and aripiprazole (3 mg/kg), and the unexpected finding that 29 of these have a reduction to some degree, and furthermore that 21 of the 32 biomarkers were reduced in concentration by at least 20% within this 60 minute period. In a similar study, where animals were treated chronically with low dose co-crystal form A (0.3 mg/kg) alone or crystal form A (0.3 mg/kg) and aripiprazole (0.3 mg/kg), the combination group had a great impact as 28/30 of the measured cytokine biomarkers of neuroinflammation were reduced, compared to 25/30 for co-crystal alone, suggesting a possible synergistic effect.

Results for TGF-j 1 levels are shown in FIG. 9.

This data show that, unexpectedly, co-crystal Form A, as well as co-crystal Form A+Aripiprazole, does not result in an increase in TGF-β levels in plasma and therefore have a lowered risk of cardiac adverse events. Unlike other drugs which bind to 5-HT2B receptors and are believed to strongly risk having cardiac adverse events, because of this. Activation of 5-HT2B receptors is associated with an increase in plasma levels of TGF-β. The present, unexpected, findings that there is no increase in TGF-β levels strongly suggest that a co-crystal of psilocybin and psilocin may significantly lower risk of causing future cardiac side-effects.

Although the invention has been described with reference to the above examples, it will be understood that modifications and variations are encompassed within the spirit and scope of the invention. Accordingly, the invention is limited only by the following claims.

1. A method of a treating a mental health disorder or a central nervous system disorder in a subject in need thereof, comprising administering to the subject a crystalline form of psilocin and psilocybin: 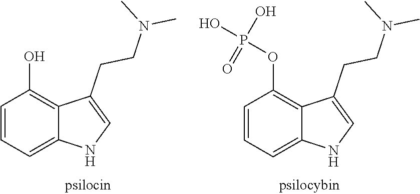
and an atypical antipsychotic.

2. The method of claim 1, wherein the crystalline form is co-crystal Form A.

3. The method of claim 2, wherein Form A is characterized by a XRPD pattern comprising a significant peak at a 2θ angle of about 10.1°.

4. The method of claim 3, wherein the XRPD pattern further comprises a significant peak at a 2θ angle of about 19.16°.

5. The method of claim 4, wherein the XRPD pattern further comprises a significant peak at a 2θ angle of about 10.74°, about 25.3°, and about 24.07°.

6. The method of claim 5, wherein the XRPD pattern further comprises a significant peak at a 2θ angle of about 14.54°, about 16.5°, about 13.44°, about 23.42°, and about 8.62°.

7-10. (canceled)

11. The method of claim 2, wherein Form A is a salt formed between psilocin and psilocybin.

12. The method of claim 1, wherein the atypical antipsychotic is aripiprazole, clozapine, ziprasidone, cariprazine, iloperidone, quetiapine, lurasidone, paliperidone, amisulpride, olanzapine, risperidone, asenapine, clozaril, brexpiprazole, lumateperone, pimavanserin, or a combination thereof.

13. The method of claim 1, wherein the atypical antipsychotic is aripiprazole.

14. The method of claim 1, wherein the mental health disorder is mood disorders, depressive disorders, mood instability disorders, mixed anxiety and depressive disorder, bipolar disorders, anxiety disorders, generalized anxiety disorders, social anxiety disorders, phobic disorders, panic attack disorders, schizophrenia, psychosis, schizoaffective disorders, eating disorders, anorexia nervosa, bulimia nervosa, substance abuse disorders, addiction disorders, alcohol abuse or dependence, opiate abuse or dependence, cocaine abuse or dependence, mixed drug abuse or dependence, behavioral addictions, gambling disorder, trauma-related disorders, post-traumatic stress disorders, grief-related disorders, loss-related disorders, end-of-life related disorders, cancer disorders, attention deficit disorder (ADD), attention deficit and hyperactivity disorder (ADHD), personality disorders, obsessive compulsive disorders, or a combination thereof.

15. The method of claim 14, wherein mental health disorder is mood disorders, mixed anxiety and depressive disorder, anxiety disorders, generalized anxiety disorders, social anxiety disorders, substance abuse disorders, addiction disorders, alcohol abuse or dependence, opiate abuse or dependence, cocaine abuse or dependence, mixed drug abuse or dependence, behavioral addictions, gambling disorder, post-traumatic stress disorders, or a combination thereof.

16. The method of claim 1, wherein the central nervous system disorder is pain disorders, Alzheimer's disease, Parkinson's Disease, a cognitive disorder, epilepsy, seizures, brain injury, stroke, aneurysm, multiple sclerosis, or a combination thereof.

17. The method of claim 1, wherein the method results in an improvement in one or more of neuroplasticity, calcium flux, 5-HT2A receptor activity, 5-HT1A, 5-HT7, TrkB activity, BDNF activity, neurogenesis, synaptogenesis, measures of second messenger functioning, or combinations thereof in the subject.

18. The method of claim 1, wherein the BDNF activity is increased.

19. (canceled)

20. The method of claim 1, wherein the method further comprises stimulating neurogenesis or neurite growth.

21-23. (canceled)

24. The method of claim 1, wherein the method comprises a decreased expression of a neuroinflammatory cytokine biomarker

25-28. (canceled)

29. The method of claim 1, wherein the method decreases the risk of cardiovascular adverse events.

30. (canceled)

31. (canceled)

32. The method of claim 1, wherein the atypical antipsychotic is administered simultaneously with the crystalline form.

33. The method of claim 1, wherein the atypical antipsychotic is administered sequentially with the crystalline form.

34-43. (canceled)
